(19) United States
(12) Patent Application Publication
(10) Pub. No.: US 2021/0198628 A1
(43) Pub. Date: Jul. 1, 2021

(54) BASAL MEDIA FOR GROWING NK-92 CELLS

(71) Applicant: NantKwest, Inc., San Diego, CA (US)

(72) Inventors: Richard John ANDERSON, San Diego, CA (US); John Charles GOULDING, San Diego, CA (US)

(21) Appl. No.: 17/057,547

(22) PCT Filed: May 20, 2019
(86) PCT No.: PCT/US2019/033066

§ 371 Date: Nov. 20, 2020

Related U.S. Application Data
(60) Provisional application No. 62/674,729, filed on May 22, 2018.

(51) Int. Cl. C12N 5/0783

(52) U.S. Cl. CPC C12N 5/0646; C12N 2500/46; C12N 2501/33; C12N 2500/34; C12N 2500/38; C12N 2501/2302; C12N 2500/50; C12N 2500/10

(57) ABSTRACT

Provided herein are methods and customized media compositions for culturing NK-92® cells.

### BACKGROUND

Natural killer (NK) cells are cytotoxic lymphocytes that constitute a major component of the innate immune system. NK cells, generally representing about 10-15% of circulating lymphocytes, bind and kill targeted cells, including virus-infected cells and many malignant cells, non-specifically with regard to antigen and without prior immune sensitization. Herberman et al., Science 214:24 (1981). Killing of targeted cells occurs by inducing cell lysis. NK cells used for this purpose are isolated from the peripheral blood lymphocyte (“PBL”) fraction of blood from the subject, expanded in cell culture in order to obtain sufficient numbers of cells, and then re-infused into the subject. NK cells have been shown to be somewhat effective in both ex vivo therapy and in vivo treatment. However, such therapy is complicated by the fact that not all NK cells are cytolytic and the therapy is specific to the treated patient.

NK-92® cells have previously been evaluated as a therapeutic agent in the treatment of certain cancers. However, the use of NK-92® cells in therapeutic applications requires consistent quantities and qualities between batches thus many of the media which use animal components such as serum are unsuitable for this purpose. Although certain media that have defined chemical compositions can be used, these media were specifically designed to support the growth of other types of cells, such as lymphokine activated killer cells. This type of media may contain unnecessary components that increase the costs of production. This type of media may also leave process residuals that affect the efficacy and purity of the NK-92® cells. Thus, a need remains for an economical growth medium that is customarily designed for growing NK-92® cells.

### BRIEF SUMMARY

Provided herein are methods and medium compositions for culturing NK-92® cells. The medium composition comprises a basal medium supplemented one or more supplements selected from the group consisting of ethanolamine (or a derivative thereof), insulin, transferrin, and human albumin (HA). Optionally, the basal medium is supplemented with sodium selenite and glucose. The basal medium itself comprises inorganic salts, vitamins and amino acids. Optionally, the basal medium itself comprises sodium selenite and glucose.

Provided herein is a method of culturing NK-92® cells comprising culturing NK-92® cells in a customized NK-92® culture medium comprising a basal medium and one or more supplements, and the one or more supplements comprise ethanolamine, an ethanolamine derivative, insulin, transferrin, sodium selenite, HA, or a combination thereof. The basal medium comprises inorganic salts, vitamins and amino acids.

Also provided herein is a method of culturing NK-92® cells comprising culturing NK-92® cells in a customized NK-92® culture medium comprising a basal medium and one or more supplements, and the one or more supplements comprise ethanolamine, an ethanolamine derivative, or a combination thereof. The basal medium comprises inorganic salts, vitamins and amino acids.

Optionally, the ethanolamine derivative is an ethanolamide. The ethanolamide may be vaccenic acid ethanolamide, or oleic acid ethanolamide, palmitic acid ethanolamide, or stearic acid ethanolamide. Optionally, the ethanolamine derivative is phosphatidylethanolamine.

Optionally, the one or more supplements comprise 1-7% human AB serum. Optionally, the one or more supplements further comprise insulin, transferrin, selenium, or a combination thereof.

Optionally the one or more supplements further comprise 300-600 IU/mL interleukin-2. Optionally the one or more supplements further comprise 0.01% to 0.1% of poloxamer 188.

Optionally the NK-92® cells cultured in the customized NK-92® culture medium have substantially the same or higher cytotoxicity, growth rate, and viability compared to NK-92® cells grown in a reference growth medium.

Optionally the NK-92® cells cultured in the customized NK-92® culture medium have 85-100% viability.

Optionally the NK-92® cells cultured in the customized NK-92® culture medium show a direct cytotoxicity of and/or an ADCC of 60-100% at an effector to target ratio of 10:1.

Optionally the NK-92® cells cultured in the customized NK-92® culture medium have a doubling time of 30-50 hours.

Optionally the NK-92® cells express a cytokine, Fc Receptor, a chimeric antigen receptor, or a combination thereof.

Optionally the customized NK-92® culture medium comprises 0.05-40 mg/L of ethanolamine, an ethanolamine derivative, or a combination thereof.

Optionally the customized NK-92® culture medium 4.5-20 g/L of glucose. Optionally the basal medium is further supplemented with 0.05% to 1.0% HA.

Optionally, the volume of the customized NK-92® culture medium is at least 5 liters.

Also provided herein is a cell culture comprising NK-92® cells in a customized NK-92® culture medium comprising a basal medium and one or more supplements, and the one or more supplements comprise ethanolamine, an ethanolamine derivative, insulin, transferrin, sodium selenite, HA, or a combination thereof, wherein the basal medium comprises inorganic salts, vitamins and amino acids.

Also provided herein is a cell culture comprising NK-92® cells in a customized NK-92® culture medium comprising a basal medium and one or more supplements, wherein the one or more supplements comprise ethanolamine, an ethanolamine derivative, or a combination thereof; and wherein the basal medium comprises inorganic salts, vitamins and amino acids. Optionally the cell culture medium is at least 5 liters.

Optionally the ethanolamine derivative is ethanolamide, wherein the ethanolamide is cis-vaccenic acid ethanolamide or oleic acid ethanolamide.

Optionally the one or more supplements further comprise insulin, transferrin, selenium, or a combination thereof. Optionally the one or more supplements further comprise 0.01% to 0.1% of poloxamer 188. Optionally the customized NK-92® culture medium comprises 4.5-20 g/L glucose. Optionally the one or more supplements comprise 0.05% to 1.0% HA.

Optionally the NK-92® cells express a cytokine, Fc Receptor, a chimeric antigen receptor, or a combination thereof.

Optionally the basal medium comprises insulin, transferrin, selenium, or a combination thereof.

Optionally the NK-92® cells have substantially the same or higher cytotoxicity, growth rate, and/or viability as compared to control NK-92® cells. Optionally the NK-92® cells that have been cultured in the customized NK-92® culture medium have 85-100% viability.

Optionally the NK-92® cells that have been cultured in the customized NK-92® culture medium have a doubling time of 30-50 hours. Optionally the NK-92® cells show a direct cytotoxicity and/or an ADCC of 60-100% when using an effector: target ratio of 10:1.

Optionally the NK-92® cells maintain substantially the same viability and/or cytotoxicity after the they are crypreserved and thawed.

The foregoing general description and the following detailed description are exemplary and explanatory and are intended to provide further explanation of the disclosure. Other objects, advantages and novel features will be readily apparent to those skilled in the art.

### BRIEF DESCRIPTION OF THE DRAWINGS

The objects, features and advantages will be more readily appreciated upon reference to the following disclosure when considered in conjunction with the accompanying drawings.

FIG. 1 is a graph showing the direct cytotoxicity of haNK® cells against K562 target cells at growth cycle 5.

### DETAILED DESCRIPTION

Provided herein are methods and medium compositions for culturing NK-92® cells. The medium composition comprises a basal medium supplemented with one or more supplements comprising ethanolamine, an ethanolamine derivative, insulin, transferrin, sodium selenite, has, or combination thereof. The basal medium itself may comprise inorganic salts, vitamins and amino acids.

After reading this description, it will become apparent to one skilled in the art how to implement various alternative embodiments and alternative applications. However, not all embodiments are described herein. It will be understood that the embodiments presented here are presented by way of an example only, and not limitation. As such, this detailed description of various alternative embodiments should not be construed to limit the scope or breadth of the disclosure as set forth herein. It is to be understood that the aspects described below are not limited to specific compositions, methods of preparing such compositions, or uses thereof as such may, of course, vary.

### Terminology

Unless defined otherwise, all technical and scientific terms used herein have the same meaning as commonly understood by one of ordinary skill in the art.

In this specification and in the claims that follow, reference will be made to a number of terms that shall be defined to have the following meanings:

The terminology used herein is for the purpose of describing particular embodiments only and is not intended to be limiting. As used herein, the singular forms “a,” “an” and “the” are intended to include the plural forms as well, unless the context clearly indicates otherwise. Thus, for example, reference to “a natural killer cell” includes a plurality of natural killer

All numerical designations, e.g., pH, temperature, time, concentration, amounts, and molecular weight, including ranges, are approximations which are varied (+) or (−) by increments of 0.1 or 1.0, where appropriate. It is to be understood, although not always explicitly stated, that all numerical designations may be preceded by the term “about.”

Unless otherwise noted, a percentage, when denoting a concentration, refer to a w/v percentage. As an example, 1.0% HA refers to the 1.0% w/v HA.

Unless otherwise noted, the concentrations in this disclosure refer to the final working concentration in the customized NK-92® culture medium.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 3, 4, or 5 cells, and so forth.

It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” is intended to mean that the compositions and methods include the recited elements, but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claims. “Consisting of” shall mean excluding more than trace amount of other ingredients and substantial method steps. Embodiments defined by each of these transition terms are within the scope of the disclosure.

As used herein, “natural killer (NK) cells” are cells of the immune system that kill target cells in the absence of a specific antigenic stimulus, and without restriction according to major histocompatibility complex (MHC) class. Target cells may be cancer or tumor cells. NK cells are characterized by the presence of CD56 and the absence of CD3 surface markers.

For purposes of this invention and unless indicated otherwise, the term “NK-92®” or “NK92” is intended to refer to the original NK-92® cell lines as well as NK-92® cell lines, clones of NK-92® cells, and NK-92® cells that have been modified (e.g., by introduction of exogenous genes). NK-92® cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92®, NK-92®-CD16, NK-92®-CD16-γ, NK-92®-CD16-ζ, NK-92®-CD16(F176V), NK-92®MI, and NK-92®CI. NK-92® cells are known to persons of ordinary skill in the art, to whom such cells are readily available from NantKwest, Inc.

As used herein, the term “aNK™ cells” refers to the parental NK-92® cells. aNK™ cells depends on 1L-2 for growth.

As used herein, the term “haNK® cells” refers to NK-92® cells that have been engineered to express Fc receptor.

As used herein, the term “taNK® cells” refers to NK-92® cells that have been engineered to express a chimeric antigen receptor (CAR) with affinity for a cancer specific antigen, a cancer associated antigen, or a tumor specific antigen. In some embodiments, the tumor specific antigen is HER-2, e.g., human HER-2, and these NK-92® cells are referred to as HER-2 taNK® cells.

As used herein, the term “t-haNK® cells” refers to NK-92® cells that have been engineered to express an Fc receptor and a chimeric antigen receptor (CAR) with affinity for a cancer specific antigen, a cancer associated antigen, or a tumor specific antigen. For example, the tumor specific antigen is CD19, and these NK-92® cells are referred to as CD19 t-haNK® cells.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FcγR) bind to the IgG class of antibodies. FcγRIII-A (also called CD16) is a low affinity Fc receptor bind to IgG antibodies and activate ADCC. FcγRIII-A are typically found on NK cells. NK-92® cells do not express FcγRIII-A.

The term “chimeric antigen receptor” (CAR), as used herein, refers to an extracellular antigen-binding domain that is fused to an intracellular signaling domain. CABs can be expressed in T cells or NK cells to increase cytotoxicity. In general, the extracellular antigen-binding domain is a scFv that is specific for an antigen found on a cell of interest. A CAR-expressing NK-92® cell is targeted to cells expressing certain antigens on the cell surface, based on the specificity of the scFv domain. The scFv domain can be engineered to recognize any antigen, including tumor-specific antigens.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, a polynucleotide encompasses both the double-stranded form and each of two complementary single-stranded forms known or predicted to make up the double-stranded form.

The term “expression” refers to the production of a gene product. The term “transient” when referred to expression means a polynucleotide is not incorporated into the genome of the cell.

The term “cytokine” or “cytokines” refers to the general class of biological molecules which effect cells of the immune system. Exemplary cytokines include, but are not limited to, interferons and interleukins (IL), in particular IL-2, IL-12, IL-15, IL-18 and IL, 21. In preferred embodiments, the cytokine is IL-2.

As used herein, the term “vector” refers to a non-chromosomal nucleic acid comprising an intact replicon such that the vector may be replicated when placed within a permissive cell, for example by a process of transformation. A vector may replicate in one cell type, such as bacteria, but have limited ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

As used herein, the term “substantially the same”, used interchangeably with the term “comparable”, or “similar”, when referring to cytotoxicity, viability or cell recovery, refers to the that the two measurements of cytotoxicity, viability or cell doubling time are no more than 25%, no more than 20%, no more than 15% different, no more than 10%, no more than 8%, or no more than 5% different from each other.

As used herein, the terms “cytotoxic” when used to describe the activity of effector cells such as NK cells, relates to killing of target cells by any of a variety of biological, biochemical, or biophysical mechanisms.

Titles or subtitles may be used in the specification for the convenience of a reader, which are not intended to influence the scope of the present disclosure. Additionally, some terms used in this specification are more specifically defined below.

### Basal Medium

This disclosure provides a culture medium customized for growing NK-92® cells (“the customized NK-92® culture medium”), which comprises a basal medium and one or more supplements, the one or more supplements comprising ethanolamine, an ethanolamine derivative, insulin, transferrin, sodium selenite, or a combination thereof. A basal medium disclosed herein refer to an unsupplemented cell culture medium. Basal medium typically contains inorganic salts, carbon source, vitamins and amino acids. Suitable basal media include but not limited to Isocove's Modified Dulbecco's Medium (IMDM) and Minimum Essential Medium Eagle alpha modification, Roswell Park Memorial Istitute (RPMT) 1640 Medium, McCoys 5A Modified Medium. Many of these media, e.g., IMDM, can be obtained commercially, for example, Thermo Fisher Scientific, Waltham, Mass., or from Sigma-Aldrich, St. Louis, Mo. Optionally, the basal culture medium comprises saccharides.

Exemplary vitamins that can be used in the basal medium may be one or more vitamins selected from the group consisting of biotin, choline chloride, D-calcium pantothenate, folic acid, niacinamide, pyridoxal hydrothloride, riboflavin, thiamine hydrochloride, vitamin B12, and i-inositol.

Exemplary inorganic salts that can be used in the basal medium may be one or more inorganic salts selected from the group consisting of CaCl₂, MgSO₄, KCl, KNO₃, NaHCO₃, NaCl, NaH₂ PO₄ and Na₂ O₃ Se.

Exemplary amino acids that can be used in the basal medium may be one or more amino acids selected from the group consisting of L-alanine, L-arginine, L-asparagine, L-aspartic acid, L-cystine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine.

Optionally, the basal medium may also comprise carbohydrates, such as saccharides including glucose. Optionally, the basal medium comprises about 3-6 g/L glucose.

Optionally, the basal medium may also comprise sodium selenite, e.g., about 10-25 ug/L, e.g., 17 ug/L.

Optionally, the basal medium may include buffer components such as 4-(2-hydroxyethyl)-1-piperazineethanesulfonic acid (HEPES). Optionally, the basal medium may include one or more other components such as phenol red, hypoxanthine monosodium salt pyruvates, linoleic acid, lipoic acid, putrescine dihydrochloride, thymidine and so forth.

Optionally, the basal medium includes sodium chloride, sodium bicarbonate, sodium phoshage monobasic, potassium chloride, calcium chloride, glucose, and HEPES.

Optionally, the basal medium comprises 2-6 g/L sodium chloride (e.g., 4-5 g/L, or 4.505 g/L sodium chloride), 1-5 g/L sodium bicarbonate (e.g., 2-4 g/L, or 3.024 g/L sodium bicarbonate), 0.1-0.6 g/L potassium chloride (e.g., 0.2-0.4 g/L, or 0.33 g/L potassium chloride), 0.1-0.4 calcium chloride (e.g., 0.15-0.2 0.1653 calcium chloride, 0.05-0.2 g/L sodium phoshage monobasic (e.g., 0.109 g/L sodium phoshage monobasic), 2-8 g/L glucose (e.g., 4.5 g/L glucose), 2-8 g/L HEPES (e.g., 5.958 g/L HEPES).

### Supplements to the Basal Medium

The customized NK-92® culture medium disclosed herein comprises the basal medium as described above plus one or more supplements, including but not limited to ethanolamine, an ethanolamine derivative, insulin, transferrin, or a combination thereof.

Albumin is a protein supplement in cell culture used to deliver unesterified fatty acids into and from cells; albumin can be, for example, human albumin (HA) or bovine serum albumin (BSA). Optionally, the albumin is human albumin or HA. Human albumin (HA) is the most copious protein in human serum at approximately 3.5-5.0 g/dL and functions as a carrier protein for steroids and fatty acids in blood. Human albumin is commercially available, for example, from CSL Behring, King of Prussia, Pa., Griffiols, Octapharma etc. Optionally, the basal medium is supplemented with 0.05-1.0% HA, e.g., 0.1-1.0%, 0.125%, 0.5%, or 1.0% of HA.

The customized NK-92® culture medium may comprise human AB serum. Human AB serum is produced by clotting human whole blood and separating the liquid phase from the clotted solid phase. As compared to HA, human AB serum contains components that are absent in HA, e.g., additional proteins (that are not used in clotting), electrolytes, antibodies, glucose, and hormones. Optionally, the basal medium is supplemented with 1-7%, e.g., 3-7%, or 5% human AB serum.

The inventors of this application have discovered that, unexpectedly, although human AB serum already contains albumin, adding low amounts of HA, e.g., 0.05-1.0%, 0.1-1.0%, or 0.125%-1%, to the basal medium that has already been supplemented with 5% human AB serum, can still significantly increase cytotoxicity of the NK-92® cells. See, e.g., Table 3 (comparing Groups E and F).

Ethanolamine is an organic chemical compound with the formula. HOCH₂CH₂NH_{2.} The molecule is both a primary amine and a primary alcohol (due to a hydroxyl group). Ethanolamine is a colorless, viscous liquid and typically used in the production of detergents, emulsifiers, polishes, pharmaceuticals, corrosion inhibitors, and chemical intermediates. Ethanolamine is also a precursor of phospho-glycerides which are essential to the structure of the plasma membrane and cellular organelles.

Ethanolamine derivatives are compounds that are derived from ethanolamine, e.g., by a chemical reaction, and comprise similar chemical structure. For example, the ethanolamine derivatives can be ethanolamides, which are formed by a condensation reaction between ethanolamine and carboxylic acids. Ethanolamine derivatives may also be an ethanolamine phospholipid, phosphatidylethanolamine. Non-limiting examples of ethanolamides that can be used include vaccenic acid ethanolamide (VEA) (e.g., cis-vaccenic acid ethanolamide), oleic acid ethanolamide (OEA) (e.g., cis-oleic acid ethanolamide), palmitic acid ethanolamide (PAE), and stearic acid ethanolamide (SEA). These compounds are fatty acid ethanolamides that typically can be found in human and rat blood plasma.

To produce the customized NK-92® culture medium, the basal medium disclosed herein may be supplemented with one or more supplements. Optionally, the one or more supplements comprise 0.2-20 mg/L ethanolamine and/or ethanolamine derivatives, e.g., 5-10 mg/L, 10-20 mg/L, 1-10 mg/L, 1-5 mg/L, or 2 mg/L.

In addition to the ethanolamine and/or its derivatives, other supplements may be used including one or more of insulin, transferrin, and selenium. insulin promotes glucose and amino acid uptake, lipogenesis, intracellular transport, and the synthesis of proteins and nucleic acids. Transferrin is an iron carrier and may also help to reduce toxic levels of oxygen radicals and peroxide. Selenium, as sodium selenite is a co-factor for glutathione peroxidase and other proteins that is used as an anti-oxidant in media. the basal medium is supplemented with a solution comprising a mixture of insulin, transferrin, selenium, and ethanolamine, Optionally, the basal medium in the customized NK-92® culture medium is supplemented with insulin, transferrin, and sodium selenite at amounts that are suitable for cell culture. For example, the basal medium may be supplemented with 1-20 mg/L, e.g., 5-10 mg/L, or 10 mg/L insulin; 2.5-12 mg/L, e.g., 5.5 mg/L transferrin, and/or 6.7 ug/L sodium selenite. Optionally, a supplement added to the basal medium to form the NK-92® culture medium is a. pre-made concentrated mixture of multiple components, such as insulin, transferrin, selenium, and ethanolamine and is diluted to a working concentration when it is added to the basal medium. The pre-made mixture may comprise, for example, 500-10,000 mg/L insulin, 250-10,000 mg/L transferrin, 0.25-15.0 mg/L sodium selenite, and 100-4,000 mg/L ethanolamine, and the pre-made mixture can be added to the basal medium at suitable dilutions, for example, 1:100, or 1:1000.

Optionally, the one or more supplements that are added to the basal medium comprise poloxamer 188 (poloxamer 188 F68). Optionally, the produced customized NK-92® culture medium comprises 0.01-0.1%, e.g., 0.05% poloxamer 188.

Optionally, the customized NK-92® culture medium comprises 4.5-20 g/L, e.g., 10-20 g/L, 5-10 g/L, about 6.5 g/L, about 9 g/L, 11 g/L, 13 g/L, 15 g/L, 17 g/L glucose.

Optionally, when the NK-92® cells are aNK™ cells, which does not produce endogenous interleukin-2 (IL-2), IL-2 is also supplemented to the basal medium at an amount sufficient to support the aNK™ cells growth. Optionally, IL-2 is supplemented to an amount of 300-600 IU/mL e.g., 500 IU/mL in the customized NK-92® culture medium.

The NK-92® cells that have been grown in the customized NK-92® culture medium have direct cytotoxicity and/or ADCC that is substantially the same as that of NK-92® cells that have been grown in a reference growth medium. Direct cytotoxicity and ADCC at different growth cycles, e.g., cycle 3, cycle 4, and/or cycle 5, may be measured using the methods described below. In some cases, the NK-92® cells that have been grown in the customized. NK-92® culture medium have direct cytotoxicity and/or ADCC that is 1-30%, e.g., 1-15%, higher than that of the NK-92® cells grown in the reference growth medium. In some cases, the NK-92® cells that been grown in the customized NK-92® culture medium have substantially the same direct cytotoxicity and/or ADCC as those of the NK-92® cells grown in the reference growth medium. An illustrative example is shown in Example 2, where Group G shows substantially the same cytotoxicity to the NK-92®) cells grown in reference culture medium.

### Exemplary Combinations of Supplements

Optionally, the customized NK-92® culture medium comprises a basal medium that is supplemented with ethanolamine but not HA. The ethanolamine may be 0.05-40 mg/L, e.g., 0.2-20 mg/L, or 2 mg/L. An illustrative example is shown in Example 2, Table 5, Groups D and F, in which aNK™ cells grown in customized NK-92® culture medium showed substantially the same growth rate, viability, and cytotoxicity as the aNK™ cells that have grown in the reference growth medium. In some cases, the basal medium is further supplemented with transferrin, insulin and sodium selenite.

Optionally, the basal medium is supplemented with ethanolamine and HA. In some cases, the HA in the customized MK-92® culture medium can be 0.05-1.0%, e.g., 0.125-1.0%, 0.25%, or 0.5%, and ethanolamine can be 2.0 mg/L. An illustrative example is shown in the Example 2., Table 5, e.g., Groups G and I.

Optionally, the basal medium is supplemented with ethanolamine, glucose, insulin, transferrin, and sodium selenite. An illustrative example is shown in Table 5, e.g., Group F.

Optionally, the basal medium is supplemented with ethanolamine, HA, and glucose. Ethanolamine and HA may be supplemented in the amounts as described above. Glucose can be added to the basal medium such that the final concentration in the customized NK-92® medium is 4.5-20 g/L. An illustrative example is shown in Table 7, e.g., Group I. Optionally, the basal medium is further supplemented with insulin, transferrin, and sodium selenite in suitable amounts disclosed above.

In one illustrative example, the basal medium comprises 4.5 g/L glucose and is supplemented with an additional amount of 2.0 g/L glucose (such that the final concentration in the customized NK-92® culture medium is 6.5 g/L), 2.0 mg/L ethanolamine, and 1.0% human albumin. In yet another illustrative example, the basal medium is supplemented with 0.2-20 mg/L ethanolamine, 0.125-0.5% of HA, and glucose in an amount such that the concentration of glucose in the customized NK-92® culture medium is about 4.5-20 g/L, e.g., 6.5 g/L. In yet another illustrative example, the basal medium comprises 4.5 g/L glucose and is supplemented with an additional amount of 2.0 g/L glucose (such that the final concentration in the customized NK-92® culture medium is 6.5 g/L), 10 mg/L insulin, 5.5 mg/L transferrin, 6.7 ug/L sodium selenite, 0.125% human albumin, and 2.0 mg/L ethanolamine.

Optionally, human AB serum and/or poloxamer 188 are also added to the basal medium that are supplemented with the various combinations of supplements described herein.

The customized NK-92® culture media comprising the basal media supplemented with the various combinations of the supplements disclosed herein support a growth rate, viability, direct cytotoxicity, and/or ADCC that is substantially the same as the NK-92® cells that have been grown in the reference growth medium.

### Other Growth Promoting Substances

Other cell growth promoting substances may also be used to supplement the basal medium for culturing NK-92® cells. These substances include, but not limited to, nucleosides, 2-ketoglutaric acid (2-oxoglutaric acid), fructose, galactose, glycerophosphoric acid, citric acid, ethanolamine, para-aminobenzoic acid, iron-containing compounds, such as FeSO₄ and hemin, benzamidine, putrescine, and unsaturated fatty acids, such as oleic acid and linolic acid.

Furthermore, in order to prevent the contamination of a medium with bacteria or mycoplasmas, antimicrobial agents, such as streptomycin, nystatin, gentamicins, ciprofloxacin, norfloxacin and levofloxacin, may be used in combination with the supplements mentioned above.

### Method of Culturing NK-92® Cells

Growing NK-92® cells typically starts from thawing frozen NK-92® cells and seeding them in a container with a suitable medium. Cells are allowed to recover until the cell viability reaches a certain value, for example, greater than 85%. Cells are then expanded in a vessel, e.g., a T-Flask or G-Rex vessel, to a desirable cell density, for example, a density that is equal to or less than 1.2×10⁶ cells/mL. The cell culture from the vessel is then collected and used to inoculate one or more larger culture vessels in the customized NK-92® culture medium as disclosed above. Commonly used such larger culture vessels include single use culture bag for a wave bioreactor, which can have a volume of at least 2 liters, at least 5 liters, at least 10 liters, or at least 25 liters. Transfer of cells between different vessels can be performed using means well known in the art, e.g., using a serological pipette, pump or a gravity feed, performed under sterile conditions.

NK-92® cells so produced can be harvested by centrifugation. Optionally, the centrifugation is performed using a continuous centrifuge aseptically attached to the culture vessel, e.g., the single use culture bags for a wave bioreactor, at the end of the expansion process. The culture supernatant is then removed and the cells are resuspended in a wash buffer. Optionally, the wash can be repeated for at least two times, at least three times, e.g., 4-6 times. After the final wash, the mixture containing the cells and wash buffer can be centrifuged again and the cells are collected and processed for therapeutic applications.

Optionally, the NK-92® cells grown in the customized NK-92® culture medium as described above are assessed for cytotoxicity. Direct cytotoxicity of the produced NK-92® cells can be assessed by methods well known in the art, for example, a ⁵¹Cr release assay (Gong et al., Leukemia, Apr; 8(4): 652-658 (1994)) using the procedure described by Klingemann et al. (Cancer Immunol. Immunother. 33:395-397 (1991)). The percentage of specific cytotoxicity can be calculated based on the amount of released ⁵¹Cr. See Patent Pub. No. US20020068044.

Alternatively, direct cytotoxicity of the produced NK-92® cells can be assessed using a calcein release assay. For example, the NK-92® cells (referred to as the effector in the assay) can be mixed with the calcein loaded target cells (referred to as target in the assay) at certain ratios. After incubation for a period of time, the calcein released from the target cells can be assessed, e.g., by a fluorescence plate reader. The ratio of the effector and target used in the assay may vary, optionally the effector: target ratio may be 20:1, 15:1, 10:1, 8:1, 5:1, 2.5:1, 1.25:1, 0.625:1, 0.31:1, 0.16:1, 0.08:1, 0.04:1, 0.02:1; optionally the effector: target ratio is 10:1. The target cells can be any cells that express MHC molecules that can be recognized by the NK-92® cells, for example, K562 cells, or BT-474 cells. The values of cytotoxicity of NK-92® cells may vary depending on the type of target cells used as well as the effector:target ratio and the growth cycle which the NK-92® cells are in. In general, the NK-92® cells produced using the methods described herein can have a direct cytotoxicity of at least 50-100%, e.g., 60-100%, 70-100%, or 80-100%. In some cases, aNK™ cells or haNK® cells may have a direct cytotoxicity of 80-110% when using K562 cells as the target cells, e.g., 82-100%, 85-100%, 87-100%, 88-100%, or 89-100%, by a calcein release assay, when the NK-92® cells are at a growth cycle selected from growth cycles 3-12, e.g., cycle 3, 4, or 5.

Optionally, the cytotoxicity of NK-92® cells, e.g., haNK® cells, that is assessed is the antibody dependent cellular cytotoxicity (ADCC). Methods for measuring the ADCC of NK-92® cells are similar to the methods of measuring direct cytotoxicity as described above except that an antibody that can recognize the target cell is added. The Fc receptor of the NK cells recognizes the cell-bound antibodies and triggers cytolytic reaction and killing the target cells. In one illustrative example, the haNK® cells can be incubated with Ramos (target cells) in the presence of Rituxan (anti-CD20 antibody) and killing of the Ramos cells can be measured by the release of internal components of the target cells, e.g., ⁵¹Cr or calcein, as described above. The ratio of the effector and target used in the assay may vary, optionally the effector: target ratio may be 20:1, 15:1, 10:1, 8:1, 5:1, 2.5:1, 1.25:1, 0.625:1, 0.31:1, 0.16:1, 0.08:1,0.04:1, or 0.02:1; preferably the effector: target ratio is 10:1. Optionally, the haNK® cells have a ADCC toxicity of at least 60%, at least 70%, at least 80%, or at least 90% when tested at an effector:target ratio of 10:1. Optionally, haNK® cells may have an ADCC cytotoxicity of 60-120%, e.g., 80-120%, 90-115%, 97-110%, or 100-120% when the haNK® cells are at a growth cycle selected from growth cycles 3-12, e.g., cycle 3, 4, or 5, when using an effector: target ratio of 10:1.

The NK-92® cells grown in the customized NK-92® culture medium may have substantially the same direct cytotoxicity and/or ADCC as those of the NK-92® cells grown in the reference growth medium. Optionally, the NK-92® cells grown in the customized NK-92® cells have a direct cytotoxicity and/or ADCC that is at 90-120%, of that of the NK-92® cells that have been grown in the reference growth medium.

The growth rate of NK-92® cells can be assessed using cell doubling time, i.e., the time it takes for the cells to proliferate and reach twice the initial cell number. The doubling time is inversely related to the growth rate of the NK-92®® cells; the greater the doubling time, the lower the growth rate. The NK-92® cells that have been grown in the customized NK-92® culture medium have a doubling time of 30-50 hours, e.g., 30-40, 40-50, or 40-47 hours.

Methods for measuring cell viability are also well known, for example, trypan blue exclusion assay, in which dead cells are stained blue and viable cell number can be calculated by substracting the trypan blue stained cells from the total cells. Cell counting can be performed on a counting chamber of a hemocytometer. Automatic cell counting, based on the fact that cells show great electrical resistance, are also commonly used to count cells as well as measure their volume. One example of the automatic cell counter is the Coulter counter, available from Beckman Coulter, Brea, CA. Another example is the NC-200™ Nucleocounter automated cell counter, available from Chemometec, Denmark. This device enumerates live and dead cells based on their staining by fluorescent viability dyes. The NK-92® cells that have grown in the customized NK-92® culture medium may have 85-100%, viability.

Optionally, the cytotoxicity, viability, and/or growth rate of the NK-92® cells grown in the media disclosed herein are compared with NK-92® cells that have been grown in a reference growth medium. The reference growth medium can be any medium that one of ordinary skill in the art have used to culture NK-92® cells. In some embodiments, the reference growth medium may comprise cytokines that are necessary for growth of certain NK-92® cells, for example, IL-2. In some cases, the reference growth medium is further supplemented with human AB serum, poloxamer 188 if the customized NK-92® culture medium is also supplemented with these ingredients. In some cases, for example, in the case of aNK™ cells the growth of which depend on interleukin-2 (IL-2), IL-2 is also supplemented in the reference growth medium. In some cases, the reference growth medium also comprises L-Serine (e.g., at a concentration of 0.324 mmol/L), L-Asparagine (e.g., at a concentration of 0.036 mmol/L), L-Glutamine (e.g., at a concentration of 0.45 mmol/L). In some cases, the reference growth medium comprises human AB serum, IL-2, and poloxamer 188. These various supplements, if used in the reference growth medium, are also supplemented to the customized NK-92® culture medium in the same study. NK-92® Cells

The NK-92® cells that can be cultured using the methods disclosed herein include aNK™ cells, haNK® taNK® and t-haNK® (e.g., CD19 t-haNK®, PD-L1 t-haNK®, or HER.2-t-haNK®) cells, which are further described below.

The NK-92® cell line is a unique cell line that was discovered to proliferate in the presence of interleukin 2 (IL-2). Gong et al., Leukemia 8:652-658 (1994). These cells have high cytolytic activity against a variety of cancers. The NK-92® cell line is a homogeneous cancerous NK cell population having broad anti-tumor cytotoxicity with predictable yield after expansion. Phase I clinical trials have confirmed its safety profile. NK-92® was discovered in the blood of a subject suffering from a non-Hodgkins lymphoma and then immortalized ex vivo. NK-92® cells are derived from NK cells, but lack the major inhibitory receptors that are displayed by normal NK cells, while retaining the majority of the activating receptors. NK-92® cells do not, however, attack normal cells nor do they elicit an unacceptable immune rejection response in humans. Characterization of the NK-92® cell line is disclosed in WO 1998/49268 and U.S. Patent Application Publication No. 2002-0068044.

The NK-92® cell line is found to exhibit the CD56^{bright}, CD2, CD7, CD11a, CD28, CD45, and CD54 surface markers. It furthermore does not display the CD1, CD3, CD4, CDS. CD8, CD10, CD14, CD16, CD19, CD20, CD23. and CD34 markers. Growth of NK-92® cells in culture is dependent upon the presence of recombinant interleukin 2 (rIL-2), with a dose as low as 1 IU/mL being sufficient to maintain proliferation. NK-92® has high cytotoxicity even at a low effector:target (E:T) ratio of 1:1. Gong, et al., supra. NK-92® cells are deposited with the American Type Culture Collection (ATCC), designation CRL-2407.

Heretofore, studies on endogenous NK cells have indicated that IL-2 (1000 IU/mL) is critical for NK cell activation during shipment, but that the cells need not be maintained at 37° C. and 5% carbon dioxide. Koepsell, et al., Transfusion 53:398-403 (2013).

Modified NK-92® cells are known and include, but are not limited to, those described in, e.g., U.S. Pat. Nos. 7,618,817, 8,034,332, and 8,313,943, U.S. Patent Application Publication No. 2013/0040386, all of which are incorporated herein by reference in their entireties, such as wild type NK-92®, NK-92®-CD16, NK-92®-CD16-γ, NK-92®-CD16-ζ, NK-92®-CD16(F157V), NK-92®mi and NK-92®ci.

Although NK-92® cells retain almost all of the activating receptors and cytolytic pathways associated with NK cells, they do not express CD16 on their cell surfaces. CD16 is an Fc receptor which recognizes and binds to the Fc portion of an antibody to activate NK cells for antibody-dependent cellular cytotoxicity (ADCC). Due to the absence of CD16 receptors, NK-92® cells are unable to lyse target cells via the ADCC mechanism and, as such, cannot potentiate the anti-tumor effects of endogenous or exogenous antibodies (i.e., rituxan and herceptin).

Studies on endogenous NK cells have indicated that IL-2 (1000 IU/mL) is critical for NK cell activation during shipment, but that the cells need not be maintained at 37° C. and 5% carbon dioxide. Koepsell, et al., Transfusion 53:398-403 (2013). However, endogenous NK cells are significantly different from NK-92® cells, in large part because of their distinct origins: NK-92® is a cancer-derived cell line, whereas endogenous NK cells are harvested from a donor (or the patient) and processed for infusion into a patient, Endogenous NK cell preparations are heterogeneous cell populations, whereas NK-92® cells are a homogeneous, clonal cell line. NK-92® cells readily proliferate in culture while maintaining cytotoxicity, whereas endogenous NK. cells do not. In addition, an endogenous heterogeneous population of NK cells does not aggregate at high density. Furthermore, endogenous NK cells express Fc receptors, including CD-16 receptors that are not expressed by NK-92® cells.

### Fc Receptors

Fc receptors bind to the Fc portion of antibodies. Several Fc receptors are known, and differ according to their preferred ligand, affinity, expression, and effect following binding to the antibody.

TABLE 1
Illustrative Fc receptors

| | Principal | Affinity | | |
|---|---|---|---|---|
| Receptor | antibody | for | | Effect following binding |
| name | ligand | ligand | Cell distribution | to antibody |
| FcγRI (CD64) | IgG1 and | High | Macrophages | Phagocytosis |
| | IgG3 | (Kd~ | Neutrophils | Cell activation |
| | | 10⁻⁹ M) | Eosinophils | Activation of respiratory |
| | | | Dendritic cells | burst |
| | | | | Induction of microbe |
| | | | | killing |
| FcγRIIA (CD32) | IgG | Low | Macrophages | Phagocytosis |
| | | (Kd > | Neutrophils | Degranulation (eosinophils) |
| | | 10⁻⁷ M) | Eosinophils | |
| | | | Platelets | |
| | | | Langerhans cells | |
| FcγRIIB1 (CD32) | IgG | Low | B Cells | No phagocytosis |
| | | (Kd > | Mast cells | Inhibition of cell activity |
| | | 10⁻⁷ M) | | |
| FcγRIIB2 (CD32) | IgG | Low | Macrophages | Phagocytosis |
| | | (Kd > | Neutrophils | Inhibition of cell activity |
| | | 10⁻⁷ M) | Eosinophils | |
| FcγRIIIA (CD16a) | IgG | Low | NK cells | Induction of antibody- |
| | | (Kd > | Macrophages (certain | dependent cell-mediated |
| | | 10⁻⁶ M) | tissues) | cytotoxicity (ADCC) |
| | | | | Induction of cytokine |
| | | | | release by macrophages |
| FcγRIIIB (CD16b) | IgG | Low | Eosinophils | Induction of microbe |
| | | (Kd > | Macrophages | killing |
| | | 10⁻⁶ M) | Neutrophils | |
| | | | Mast cells | |
| | | | Follicular dendritic | |
| | | | cells | |
| FcεRI | IgE | High | Mast cells | Degranulation |
| | | (Kd~ | Eosinophils | Phagocytosis |
| | | 10⁻¹⁰ M) | Basophils | |
| | | | Langerhans cells | |
| | | | Monocytes | |
| FcεRII (CD23) | IgE | Low | B cells | Possible adhesion molecule |
| | | (Kd > | Eosinophils | IgE transport across human |
| | | 10⁻⁷ M) | Langerhans cells | intestinal epithelium |
| | | | | Positive-feedback |
| | | | | mechanism to enhance |
| | | | | allergic sensitization (B |
| | | | | cells) |
| FcαRI (CD89) | IgA | Low | Monocytes | Phagocytosis |
| | | (Kd > | Macrophages | Induction of microbe |
| | | 10⁻⁶ M) | Neutrophils | killing |
| | | | Eosinophils | |
| Fcα/μR | IgA and IgM | High for | B cells | Endocytosis |
| | | IgM, | Mesangial cells | Induction of microbe |
| | | Mid for | Macrophages | killing |
| | | IgA | | |
| FcRn | IgG | | Monocytes | Transfers IgG from a |
| | | | Macrophages | mother to fetus through the |
| | | | Dendritic cells | placenta |
| | | | Epithelial cells | Transfers IgG from a |
| | | | Endothelial cells | mother to infant in milk |
| | | | Hepatocytes | Protects IgG from |
| | | | | degradation |

Optionally, NK-92® cells are modified to express an Fc receptor protein on the cell surface.

Optionally, the Fc receptor is CD16. A representative amino acid sequence encoding CD16 is shown in SEQ ID NO:2. A representative polynucleotide sequence encoding CD16 is shown in SEQ D NO:1. In some embodiments, NK-92® cells are modified by introducing a polynucleotide encoding a CD16 polypeptide has at least about 70% polynucleotide sequence identity with a polynucleotide sequence encoding a full-length, including signal peptide, naturally occurring CD16 that has a phenylalanine at position 176 of the full-length CD1.6. Optionally, a polynucleotide encoding a CD16 polypeptide has at least about 70% polynucleotide sequence identity with a polynucleotide sequence encoding a full-length, including the signal peptide, naturally occurring CD16 that has a valine at position 176.

Homologous polynucleotide sequences include those that encode polypeptide sequences coding for variants of CD16. Optionally, homologous CD16 polynucleotides may be about 150 to about 700, about 750, or about 800 polynucleotides in length, although CD16 variants having more than 700 to 800 polynucleotides are within the scope of the disclosure.

In other examples, cDNA sequences having polymorphisms that change the CD16 amino acid sequences are used to modify the NK-92® cells, such as, for example, the allelic variations among individuals that exhibit genetic polymorphisms in CD16 genes. In other examples, CD16 genes from other species that have a polynucleotide sequence that differs from the sequence of human CD16 are used to modify NK-92® cells.

In examples, variant polypeptides are made using methods known in the art such as oligonucleotide-mediated (site-directed) mutagenesis, alanine scanning, and PCR mutagenesis. Site direct mutagenesis (Carter, 1986; Zoller and Smith, 1987), cassette mutagenesis, restriction selection mutagenesis (Wells et al,, 1985) or other known techniques can be performed on the cloned DNA to produce CD16 variants (Ausubel, 2002; Sambrook and Russell, 2001),

Conservative substitutions in the amino acid sequence of human CD16 polypeptide, whereby an amino acid of one class is replaced with another amino acid of the same class, fall within the scope of the disclosed CD16 variants as long as the substitution does not materially alter the activity of the polypeptide. Conservative substitutions are well known to one of skill in the art. Non-conservative substitutions that affect(1) the structure of the polypeptide backbone, such as a β-sheet or α-helical conformation, (2) the charge, (3) the hydrophobicity, or (4) the bulk of the side chain of the target site can modify CD16 polypeptide function or immunological identity. Non-conservative substitutions entail exchanging a member of one of these classes for another class. Substitutions may be introduced into conservative substitution sites or more preferably into non-conserved sites.

Optionally, CD16 polypeptide variants are at least 200 amino acids in length and have at least 70% amino acid sequence identity, or at least 80%, or at least 90% identity to SEQ ID NO:1 or SEQ ID NO:2. In some embodiments, CD16 polypeptide variants are at least 225 amino acid in length and have at least 70% amino acid sequence identity, or at least 80%, or at least 90% identity to SEQ ID NO:1 or SEQ ID NO:2.

In some embodiments a nucleic acid encoding a CD16 polypeptide may encode a CD16 fusion protein. A CD16 fusion polypeptide includes any portion of CD16 or an entire CD16 fused with a non-CD16 polypeptide. In some embodiment, a fusion polypeptide may be created in which a heterologous polypeptide sequence is fused to the C-terminus of CD16 or is positioned internally in the CD16. Typically, up to about 30% of the CD16 cytoplasmic domain may be replaced. Such modification can enhance expression or enhance cytotoxicity (e.g., ADCC responsiveness). In other examples, chimeric proteins, such as domains from other lymphocyte activating receptors, including but not limited to Ig-a, CD3-e, CD3-d, DAP-12 and DAP-10, replace a portion of the CD16 cytoplasmic domain.

Fusion genes can be synthesized by conventional techniques, including automated DNA synthesizers and PCR amplification using anchor primers that give rise to complementary overhangs between two consecutive gene fragments that can subsequently be annealed and re-amplified to generate a chimeric gene sequence (Ausubel, 2002). Many vectors are commercially available that facilitate sub-cloning CD16 in-frame to a fusion moiety

### Chimeric Antigen Receptor

As described herein, NK-92® cells are further engineered to express a chimeric antigen receptor (CAR) on the cell surface. Optionally, the CAR is specific for a tumor-specific antigen. Tumor-specific antigens are described, by way of non-limiting example, in U.S. 2013/0189268; WO 1999024566 A1; U.S. Pat. No. 7,098,008; and WO 2000020460 A1, each of which is incorporated herein by reference in its entirety. Tumor-specific antigens include, without limitation, NKG2D, CS1, GD2, CD138, EpCAM, EBNA3C, GPA7, CD244, CA-125, ETA, MAGE, CAGE, RAGE, RAGE, LACE, PAGE, NY-SEO-1, GAGE, CEA, CD52, CD30, MUC5AC, c-Met, EGFR, FAB, WT-1, PSMA, NY-ESO1, AFP, CEA, CTAG1B, CD19 and CD33. Additional non-limiting tumor-associated antigens, and the malignancies associated therewith, can be found in Table 2.

TABLE 2
Tumor-Specific Antigens and Associated Malignancies

| Target Antigen | Associated Malignancy |
|---|---|
| α-Folate Receptor | Ovarian Cancer |
| CAIX | Renal Cell Carcinoma |
| CD19 | B-cell Malignancies |
| | Chronic lymphocytic leukemia (CLL) |
| | B-cell CLL (B-CLL) |
| | Acute lymphoblastic leukemia (ALL); ALL |
| | post Hematopoietic stem cell transplantation |
| | (HSCT) |
| | Lymphoma; Refractory Follicular |
| | Lymphoma; B-cell non-Hodgkin lymphoma |
| | (B-NHL) |
| | Leukemia |
| | B-cell Malignancies post-HSCT |
| | B-lineage Lymphoid Malignancies post |
| | umbilical cord blood transplantation (UCBT) |
| CD19/CD20 | Lymphoblastic Leukemia |
| CD20 | Lymphomas |
| | B-Cell Malignancies |
| | B-cell Lymphomas |
| | Mantle Cell Lymphoma |
| | Indolent B-NHL |
| | Leukemia |
| CD22 | B-cell Malignancies |
| CD30 | Lymphomas; Hodgkin Lymphoma |
| CD33 | AML |
| CD44v7/8 | Cervical Carcinoma |
| CD138 | Multiple Myeloma |
| CD244 | Neuroblastoma |
| CEA | Breast Cancer |
| | Colorectal Cancer |
| CS1 | Multiple Myeloma |
| EBNA3C | EBV Positive T-cells |
| EGP-2 | Multiple Malignancies |
| EGP-40 | Colorectal Cancer |
| EpCAM | Breast Carcinoma |
| Erb-B2 | Colorectal Cancer |
| | Breast Cancer and Others |
| | Prostate Cancer |
| Erb-B 2,3,4 | Breast Cancer and Others |
| FBP | Ovarian Cancer |
| Fetal | Rhabdomyosarcoma |
| Acetylcholine Receptor | |
| GD2 | Neuroblastoma |
| GD3 | Melanoma |
| GPA7 | Melanoma |
| Her2 | Breast Carcinoma |
| | Ovarian Cancer |
| | Tumors of Epithelial Origin |
| Her2/new | Medulloblastoma |
| | Lung Malignancy |
| | Advanced Osteosarcoma |
| | Glioblastoma |
| IL-13R-a2 | Glioma |
| | Glioblastoma |
| | Medulloblastoma |
| KDR | Tumor Neovasculature |
| k-light chain | B-cell Malignancies |
| | B-NHL, CLL |
| LeY | Carcinomas |
| | Epithelial Derived Tumors |
| L1 Cell Adhesion Molecule | Neuroblastoma |
| MAGE-A1 | Melanoma |
| Mesothelin | Various Tumors |
| MUC1 | Breast Cancer; Ovarian Cancer |
| NKG2D Ligands | Various Tumors |
| Oncofetal Antigen (h5T4) | Various Tumors |
| PSCA | Prostate Carcinoma |
| PSMA | Prostate/Tumor Vasculature |
| TAA Targeted by mAb IgE | Various Tumors |
| TAG-72 | Adenocarcinomas |
| VEGF-R2 | Tumor Neovasculature |

Optionally, the CAR targets CD19, CD33 or CSPG-4.

In examples, variant polypeptides are made using methods known in the art such as oligonucleotide-mediated (site-directed) mutagenesis, alanine scanning, and PCR mutagenesis. Site direct mutagenesis (Carter, 1986; Zoller and Smith, 1987), cassette mutagenesis, restriction selection mutagenesis (Wells et al,, 1985) or other known techniques can be performed on the cloned DNA to produce CD16 variants (Ausubel, 2002; Sambrook and Russell, 2001).

Optionally, the CAR targets an antigen associated with a specific cancer type. Optionally, the cancer is selected from the group consisting of leukemia (including acute leukemias (e.g., acute lymphocytic leukemia, acute myelocytic leukemia (including myeloblastic, promyelocytic, myelomonocytic, monocytic, and erythroleukemia)) and chronic leukemias e.g., chronic myelocytic granulocytic) leukemia and chronic lymphocytic leukemia polycythemia vera, lymphomas (e.g., Hodgkin's disease and non-Hodgkin's disease), multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, a polynucleotide encoding a CAR is mutated to alter the amino acid sequence encoding for CAR without altering the function of the CAR. For example, polynucleotide substitutions leading to amino acid substitutions at “non-essential” amino acid residues can be made in the CARs disclosed above. CARs can be engineered as described, for example, in Patent Publication Nos. WO 2014039523; US 20140242701; US 20140274909; US 20130280285; and WO 2014099671, each of which is incorporated herein by reference in its entirety. Optionally, the CAR is a CD19 CAR, a CD33 CAR or CSPG-4 CAR.

### Additional Modifications—Cytokines

The cytotoxicity of NK-92® cells is dependent on the presence of cytokines (e.g., interleukin-2 (IL-2). The cost of using exogenously added IL-2 needed to maintain and expand. NK-92® cells in commercial scale culture is significant. The administration of IL-2 human subjects in sufficient quantity to continue activation of NK-92® cells would cause adverse side effects.

Optionally, RR-expressing NK-92® cells are further modified to express at least one cytokine and a suicide gene. In specific embodiments, the at least one cytokine is IL-2, IL-12, IL-15, IL-18, 1L-21 or a variant thereof. In preferred embodiments, the cytokine is IL-2. A representative nucleic acid encoding IL-2 is shown in SEQ ID NO:3 and a representative polypeptide of 1L-2 is shown in SEQ ID NO:4. In certain embodiments the IL-2 is a variant that is targeted to the endoplasmic reticulum.

In one embodiment, the IL-2 is expressed with a signal sequence that directs the IL-2 to the endoplasmic reticulum. Not to be bound by theory, but directing the IL-2 to the endoplasmic reticulum permits expression of IL-2 at levels sufficient for autocrine activation, but without releasing IL-2 extracellularly. See Konstantinidis et al “Targeting IL-2 to the endoplasmic reticulum confines autocrine growth stimulation to NK-92® cells” Exp. Hematol. 2005 February;33(2):159-64. Continuous activation of the RR-expressing NK-92® cells can be prevented, e.g., by the presence of the suicide gene.

### Additional Modifications—Suicide Gene

The term “suicide gene” is one that allows for the negative selection of the cells. A suicide gene is used as a safety system, allowing the cells expressing the gene to be killed by introduction of a selective agent. This is desirable in case the recombinant gene causes a mutation leading to uncontrolled cell growth. A number of suicide gene systems have been identified, including the herpes simplex virus thymidine kinase (TK) gene, the cytosine deaminase gene, the varicella-zoster virus thymidine kinase gene, the nitroreductase gene, the Escherichia coli gpt gene, and the E. coli Deo gene (also see, for example, Yazawa K, Fisher W E, Brunicardi F C: Current progress in suicide gene therapy for cancer. World J. Surg. 2002 July; 26(7):783-9). As used herein, the suicide gene is active in NK-92® cells. Typically, the suicide gene encodes for a protein that has no ill-effect on the cell but, in the presence of a specific compound, will kill the cell. Thus, the suicide gene is typically part of a system.

In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir.

In another embodiment, the suicide gene is Cytosine deaminase which is toxic to cells in the presence of 5-fluorocytosine. Garcia-Sanchez et al. “Cytosine deaminase adenoviral vector and 5-fluorocytosine selectively reduce breast cancer cells 1 million-fold when they contaminate hematopoietic cells: a potential purging method for autologous transplantation.” Blood 1998 July 15;92(2):672-82.

In another embodiment, the suicide gene is cytochrome P450 which is toxic in the presence of ifosfamide, or cyclophosphamide. See e.g., Touati et al. “A suicide gene therapy combining the improvement of cyclophosphamide tumor cytotoxicity and the development of an anti-tumor immune response.” Curr Gene Ther. 2014;14(3):236-46.

In another embodiment, the suicide gene is iCas9. Di Stasi, (2011) “inducible apoptosis as a safety switch for adoptive cell therapy.” N Engl J Med 365: 1673-1683. See also Morgan, “Live and Let Die: A New Suicide Gene Therapy Moves to the Clinic” Molecular Therapy (2012); 20: 11-13. The iCas9 protein induces apoptosis in the presence of a small molecule AP1903. AP1903 is biologically inert small molecule, that has been shown in clinical studies to be well tolerated, and has been used in the context of adoptive cell therapy.

In one embodiment, the modified NK-92® cells are irradiated prior to administration to the patient. Irradiation of NK-92® cells is described, for example, in U.S. Pat. No. 8,034,332, which is incorporated herein by reference in its entirety. In one embodiment, modified NK-92® cells that have not been engineered to express a suicide gene are irradiated.

### Transgene Expression

Transgenes (e.g., CD19.CAR and CDI6) can be engineered into an expression vector by any mechanism known to those of skill in the art. Transgenes may be engineered into the same expression vector or a different expression vector. In preferred embodiments, the transgenes are engineered into the same vector.

In some embodiments, the vector allows incorporation of the transgene(s) into the genome of the cell. In some embodiments, the vectors have a positive selection marker. Positive selection markers include any genes that allow the cell to grow under conditions that would kill a cell not expressing the gene. Non-limiting examples include antibiotic resistance, e.g., geneticin (Neo gene from Tn5).

Any number of vectors can be used to express the Fc receptor and/or the CAR. In some embodiments, the vector is a plasmid. In one embodiment, the vector is a viral vector. Viral vectors include, but are not limited to, retroviral vectors, adenoviral vectors, adeno-associated viral vectors, herpes simplex viral vectors, pox viral vectors, and others.

Transgenes can be introduced into the NK-92® cells using any transfection method known in the art, including, by way of non-limiting example, infection, electroporation, lipofection, nucleofection, or “gene-gun.”

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EMBODIMENTS

The methods and compositions disclosed herein include the following exemplary embodiments.

Embodiment 1. A method of culturing NK-92® cells comprising culturing NK-92® cells in a customized NK-92® culture medium comprising a basal medium and one or more supplements, wherein the one or more supplements comprise ethanolamine, an ethanolamine derivative, insulin, transferrin, sodium selenite. HA, or a combination thereof, and wherein the basal medium comprises inorganic salts, vitamins and amino acids.

Embodiment 2. The method of embodiment 1, wherein the ethanolamine derivative is an ethanolamide.

Embodiment 3. The method of embodiment 2, wherein the ethanolamide is vaccenic acid ethanolamide, or oleic acid ethanolamide, palmitic acid ethanolamide, or stearic acid ethanolamide.

Embodiment 4. The method of embodiment 1, wherein the ethanolamine derivative is phosphatidylethanolamine.

Embodiment 5. The method of any of embodiments 1-4, wherein the one or more supplements comprise 1-7% human AB serum.

Embodiment 6. The method of any of embodiments 1-5, wherein the one or more supplements further comprise insulin, transferrin, selenium, or a combination thereof.

Embodiment 7. The method of any of embodiments 1-6, wherein the one or more supplements further comprise 300-600 IU/mL interleukin-2.

Embodiment 8. The method of any of embodiments 1-7, wherein the one or more supplements further comprise 0.01% to 0.1% of poloxamer 188.

Embodiment 9. The method of any of embodiments 1-8, wherein the NK-92® cells cultured in the customized NK-92® culture medium have substantially the same or higher cytotoxicity, growth rate, and viability compared to NK-92® cells grown in a reference growth medium.

Embodiment 10. The method of any of embodiments 1-9, wherein the NK-92® cells cultured in the customized NK-92® culture medium have 85-100% viability.

Embodiment 11. The method of any of embodiments 1-10, wherein the NK-92® cells cultured in the customized NK-92® culture medium show a direct cytotoxicity of and/or an ADCC of 60-100% at an effector to target ratio of 10:1.

Embodiment 12. The method of any of embodiments 1-11, wherein the NK-92® cells cultured in the customized NK-92® culture medium have a doubling time of 30-50 hours.

Embodiment 13. The method of any of embodiments 1-12, wherein the NK-92® cells express a cytokine, Fc Receptor, a chimeric antigen receptor, or a combination thereof.

Embodiment 14. The method of embodiments 1-13, wherein the customized NK-92® culture medium comprises 0.05-40 mg/L of ethanolamine, an ethanolamine derivative, or a combination thereof.

Embodiment 15. The method of embodiments 1-14, wherein the customized NK-92® culture medium 4.5-20 g/L of glucose.

Embodiment 16. The method of any of embodiments 1-15, wherein the customized NK-92® culture medium is further supplemented with 0.05% to 1.0% HA.

Embodiment 17. A cell culture comprising NK-92® cells in a customized NK-92® culture medium comprising a basal medium and one or more supplements, wherein the one or more supplements comprise ethanolamine, an ethanolamine derivative, insulin, transferrin, sodium selenite, HA, or a combination thereof; and wherein the basal medium comprises inorganic salts, vitamins and amino acids.

Embodiment 18. A cell culture comprising NK-92 cells in a customized NK-922′ culture medium comprising a basal medium and one or more supplements, wherein the one or more supplements comprise ethanolamine, an ethanolamine derivative, or a combination thereof; and wherein the basal medium comprises inorganic salts, vitamins and amino acids.

Embodiment 19. The cell culture of embodiment 17 or 18, wherein the ethanolamine derivative is ethanolamide.

Embodiment 20. The cell culture of embodiment 19, wherein the ethanolamide is cis-vaccenic acid ethanolamide or oleic acid ethanolamide.

Embodiment 21. The cell culture of embodiment 18, wherein one or more supplements further comprise insulin, transferrin, selenium, or a combination thereof.

Embodiment 22. The cell culture of any of embodiments 17-21, wherein the one or more supplements further comprise 0.01% to 0.1% of poloxamer 188.

Embodiment 23. The cell culture of any of embodiments 17-22, wherein the customized NK-92® culture medium comprises 4.5-20 g/L glucose.

Embodiment 24. The cell culture of any of embodiments 17-23, wherein the one or more supplements comprise 0.05% to 1.0% HA.

Embodiment 25. The cell culture of any of embodiments 17-24, wherein the NK-92® cells express a cytokine, Fc Receptor, a chimeric antigen receptor, or a combination thereof.

Embodiment 26. The cell culture of any of embodiments 17-25, wherein the customized NK-92® culture medium comprises insulin, transferrin, selenium, or a combination thereof.

Embodiment 27. The cell culture of any of embodiments 17-26, wherein the NK-92® cells have substantially the same or higher cytotoxicity, growth rate, and/or viability as compared to control NK-92® cells.

Embodiment 28. The cell culture of any of embodiments 17-27, wherein the NK-92® cells that have been cultured in the customized NK-92® culture medium have 85-100% viability.

Embodiment 29. The cell culture of any of embodiments 17-28, wherein the NK-92® cells that have been cultured in the customized NK-92® culture medium the doubling time of the NK-92® cells is 30-50 hours.

Embodiment 30. The cell culture of any of embodiments 17-29, wherein the NK-92® cells show a direct cytotoxicity and/or an ADCC of 60-100% when using an effector: target ratio of 10:1.

Embodiment 31. The method of any of the embodiments 1-16, wherein the volume of the customized NK-92® culture medium is at least 5 liters.

Embodiment 32. The cell culture of any of the embodiments 17-30, wherein the cell culture volume is at least 5 liters.

Embodiment 33. The cell culture of any of the embodiments 17-30, wherein the NK-92® cells maintain substantially the same viability and/or cytotoxicity after the cells are crypreserved and thawed.

### EXAMPLES

The following examples are for illustrative purposes only and should not be interpreted as limitations. There are a variety of alternative techniques and procedures available to those of skill in the art which would similarly permit one to successfully perform the examples below.

### Example 1: Effect of Ethanolamine and HA Supplementation on aNK™ Cells

Growth of aNK™ cells in IMDM based medium (a basal medium) containing different supplements was compared with aNK™ cells grown in a reference growth medium.

The interleukin-2 (IL-2)-dependent NK-92® parental cell line (aNK™) is presently cultured in a reference growth medium supplemented with human AB serum and IL-2. In this study, it was determined whether Iscove's Modified Dulbeco's Medium (IMDM) supplemented with human AB serum and IL-2 and additional supplements can support the growth, viability and functionreal activity of the aNK™ cell line in a similar manner to the reference growth medium.

The growth, viability and function of the aNK™ cell line cultured in IMDM basal media containing human AB serum and IL-2 and additional supplements (Table 3) was compared with the reference growth medium. The aNK™ cell line was cultured in each media formulation for five growth cycles of 3 to 4 days in T75 tissue culture flasks. For each growth cycle, the cells were grown from ˜0.3×10⁶ cells/mL to ˜10⁶ cells/mL per growth cycle. Growth rate and viability was determined by automated cell counting at the time of seeding and harvest. Functional activity was measured as cytotoxicity against K562 cells using a Calcein AM release assay following standard methods at the end of the third, fourth and fifth growth cycles.

Table 3 shows the formulations of various media used in this study.

TABLE 3
Media Formulations

| Grp | Medium¹ | Additional Supplements | Abbreviation |
|---|---|---|---|
| A | Reference | None | None |
| | growth | | |
| | medium | | |
| B | IMDM | None | IMDM |
| C | IMDM | 10.0 mg/L Insulin, 5.5 mg/L Transferrin, 6.7 | IMDM, insulin, |
| | | ug/L Sodium Selenite² | transferrin, |
| | | | selenium |
| D | IMDM | 10.0 mg/L Insulin, 5.5 mg/L Transferrin, 6.7 | IMDM, insulin, |
| | | ug/L Sodium Selenite², 2.0 mg/L | transferrin, |
| | | Ethanolamine | selenium, |
| | | | ethanolamine |
| E | IMDM | 10 mg/L Insulin, 5.5 mg/L Transferrin, 6.7 | IMDM, insulin, |
| | | ug/L Sodium Selenite², 2.0 mg/L | transferrin, |
| | | Ethanolamine, 1.0% Human Albumin | selenium, |
| | | | ethanolamine, HA |
| F | IMDM | 10 mg/L Insulin, 5.5 mg/L Transferrin, 6.7 | IMDM, insulin, |
| | | ug/L Sodium Selenite², 2.0 mg/L | transferrin, |
| | | Ethanolamine, 2.0 g/L Glucose³ | selenium, |
| | | | ethanolamine, |
| | | | Glucose |
| G | IMDM | 10 mg/L Insulin, 5.5 mg/L Transferrin, 6.7 | IMDM, insulin, |
| | | ug/L Sodium Selenite², 2.0 mg/L | transferrin, |
| | | Ethanolamine, 2.0 g/L Glucose³, 1.0% Human | selenium, |
| | | Albumin | ethanolamine, |
| | | | Glucose, HA |
| H | IMDM | 10 mg/L Insulin, 5.5 mg/L Transferrin, 6.7 | Imdm, insulin, |
| | | ug/L Sodium Selenite², 2.0 g/L Glucose³ | transferrin, |
| | | | selenium, glucose |
| I | IMDM | 10 mg/L Insulin, 5.5 mg/L Transferrin, | Imdm, insulin, |
| | | 2.0 g/L Glucose³, 1.0% Human Albumin | transferrin, |
| | | | selenium, glucose, |
| | | | HA |

¹All media were supplemented with Human AB Serum and Poloxamer 188.
²Sodium Selenite is present in the IMDM basal media formulation at 17 μg/L. The media was supplemented with 6.7 μg/L Sodium Selenite to give a final concentration of 23.7 μg/L Sodium Selenite.
³Glucose is present in the IMDM basal media formulation at 4.5 g/L. The media was supplemented with 2 g/L glucose to give a final concentration of 6.5 g/L glucose.

In this study, both media were supplemented with human AB serum and interleukin-2. Sodium selenite was present in the IMDM basal media formulation at 17 μg/L. The media was supplemented with 6.7 μg/L sodium selenite to give a final concentration of 23.7 μg/L sodium selenite. Glucose was present in the IMDM basal media formulation at 4.5 g/L. The media was supplemented with 2 g/L glucose to give a final concentration of 6.5 g/L glucose.

The results are shown in Table 4.

TABLE 4
Study Results

| | | | Average ± | | | | |
|---|---|---|---|---|---|---|---|
| | | | SD | Average ± | Average ± SD % | | |
| | | | Doubling | SD | Cytotoxicity⁴ against K562 | | |
| | | Additional | Time | Viability | at E:T 10 | | |
| Grp. | Medium¹ | Supplements | (hours)² | (%)³ | Cycle 3 | Cycle 4 | Cycle 5 |
| A | Reference | — | 39.7 ± 1.6 | 94.8 ± 1.5 | 90 ± 5 | 85 ± 3 | 91 ± 4 |
| | growth | | | | | | |
| | medium | | | | | | |
| B | IMDM | — | 44.6 ± 3.9 | 91.8 ± 2.3 | 63 ± 3 | 63 ± 3 | 80 ± 6 |
| C | IMDM | insulin, | 43.1 ± 2.4 | 92.3 ± 2.8 | 54 ± 2 | 65 ± 5 | 83 ± 0 |
| | | transferrin, | | | | | |
| | | selenium | | | | | |
| D | IMDM | insulin, | 41.6 ± 3.9 | 92.9 ± 1.7 | 85 ± 2 | 88 ± 4 | 98 ± 9 |
| | | transferrin, | | | | | |
| | | selenium, | | | | | |
| | | ethanolamine | | | | | |
| E | IMDM | insulin, | 48.6 ± 5.4 | 93.7 ± 1.4 | 97 ± 5 | 95 ± 6 | 104 ± 3 |
| | | transferrin, | | | | | |
| | | selenium, | | | | | |
| | | ethanolamine, | | | | | |
| | | HA | | | | | |
| F | IMDM | insulin, | 40.5 ± 3.8 | 91.9 ± 1.8 | 89 ± 2 | 93 ± 5 | 94 ± 5 |
| | | transferrin, | | | | | |
| | | selenium, | | | | | |
| | | ethanolamine, | | | | | |
| | | glucose | | | | | |
| G | IMDM | insulin, | 40.9 ± 4.2 | 94.1 ± 2.0 | 97 ± 3 | 95 ± 5 | 96 ± 3 |
| | | transferrin, | | | | | |
| | | selenium, | | | | | |
| | | ethanolamine, | | | | | |
| | | glucose, HA | | | | | |
| H | IMDM | insulin, | 43.0 ± 3.4 | 92.5 ± 1.9 | 62 ± 2 | 65 ± 1 | 76 ± 3 |
| | | transferrin, | | | | | |
| | | selenium, | | | | | |
| | | glucose | | | | | |
| I | IMDM | insulin, | 46.3 ± 7.4 | 92.9 ± 2.0 | 88 ± 2 | 98 ± 4 | 90 ± 2 |
| | | transferrin, | | | | | |
| | | selenium, | | | | | |
| | | glucose, HA | | | | | |

¹All media were supplemented with Human AB Serum and Interleukin-2 in addition to the supplements shown in the table.
²Average ± standard deviation doubling time was calculated for five growth cycles from viable cell density (VCD) of the aNK ™ cell culture determined at the start and end of each growth cycle using an NC200 automated cell counter using the following equation: Ln2/(Ln (VCD End of Cycle/VCD Start of Cycle)/Length of Cycle)).
³Average ± standard deviation viability was calculated for five growth cycles from the cell viability of the aNK ™ cell cultures determined at the start and end of each growth cycle using an NC-200 automated cell counter.
⁴Cytotoxicity against K562 cells was determined using a Calcein release assay following standard methods.

In this study, all basal media were supplemented with human AB serum and interleukin-2 in addition to the supplements shown in the table. The average±standard deviation doubling time was calculated for five growth cycles from viable cell density (VCD) of the aNK™ cell culture determined at the start and end of each growth cycle using an NC-200 automated cell counter using the following equation: Ln2/(Ln (VCD End of Cycle/VCD Start of Cycle)/Length of Cycle)). Average±standard deviation viability was calculated for five growth cycles from the cell viability of the aNK™ cell cultures determined at the start and end of each growth cycle using an NC-200 automated cell counter. Cytotoxicity against K562 cells was determined using a Calcein release assay following standard methods.

As shown in Table 4, while the aNK™ cell line grew in the IMDM basal media without specific supplements (Group B), the doubling time and viability was greater than that exhibited by the control cells grown aNK™ reference growth medium (Group A). Furthermore, the cytotoxicity of the aNK™ cells was substantially deficient following growth in the IMDM basal media alone (Group B versus Group A).

Supplementation of the IMDM basal media with only 10.0 mg/L insulin, 5.5 mg/L transferrin, 6.7 μg/L sodium selenite (Group C and H) did not restore the cytotoxicity of the aNK™ cells. However, cytotoxicity was restored by the addition of 10.0 mg/L insulin, 5.5 mg/L transferrin, 6.7 μg/L sodium selenite and 2.0 mg/L ethanolamine to the media (Groups D and F). Cytotoxicity was further enhanced by the addition of 1% human albumin to the IMDM basal formulation containing insulin, transferrin, sodium selenite and ethanolamine (Group E). Increasing the glucose concentration in this formulation from 4.5 g/L to 6.5 g/L increased the growth rate of the aNK™ cells (Group G). Notably, the cytotoxicity of the aNK™ cell line cultured in this optimal IMDM formulation was superior to that of aNK™ reference growth medium.

Absence of ethanolamine from this formulation increased the average doubling time and marginally decreased the viability (Group I versus Group G). Furthermore, the functional activity was reduced on Cycles 3 and 5 in the group that did not contain ethanolamine (Group I versus Group G).

The data shown in this study indicate that IMDM basal media supplemented with 10 mg/L insulin. 5.5 mg/L transferrin, 6.7 μg/L sodium selenite, 2.0 mg/L ethanolamine, 2.0 g/L glucose, 1.0% human albumin supported an equivalent growth rate and viability, and superior functional activity of aNK.TM compared to reference growth medium containing human AB serum.

The data show ethanolamine enhances the functional activity of aNK™ in the absence of 1.0% human albumin supplementation in the IMDM basal media formulation and ethanolamine enhanced the growth rate of aNK™ and marginally enhanced the functional activity of aNK™ cells in the presence of 1% human albumin.

### Example 2: Effect of HA and Ethanolamine Supplementation (haNK® Cell Study)

In this example, NK-92® reference growth medium or IMDM supplemented with various components were used to grow haNK® cells. In addition to the components in the Table 4, all media were supplemented with human AB serum and poloxamer 188. The average doubling time, average viability, direct cytotoxicity, and antibody dependent cellular cytotoxicity (ADCC) at the third growth cycle (cycle 3) and at the fifth growth cycle (cycle 5) were assessed and shown in Table 5.

TABLE 5
Ethanolamine and Insulin, Transferrin, Selenium and Ethanolamine

| | | | Average | | Direct | | | |
|---|---|---|---|---|---|---|---|---|
| | | | Doubling | Average | Cytotoxicity | | ADCC (%) | |
| | | | Time | Viability | (%) at E:T 10:1 | | at E:T 10:1 | |
| Grp. | Medium¹ | Supplements | (hours) | (%) | Cycle 3 | Cycle 5 | Cycle 3 | Cycle 5 |
| A | Reference | — | 36.2 ± 1.4 | 94.9 ± 2.6 | 72 ± 3 | 63 ± 3 | 97 ± 6 | 87 ± 1 |
| | growth | | | | | | | |
| | medium | | | | | | | |
| B | IMDM | Insulin, | 44.9 ± 13.2 | 93.9 ± 1.9 | 59 ± 4 | 57 ± 3 | 95 ± 11 | 94 ± 4 |
| | | Transferrin, | | | | | | |
| | | Selenium, | | | | | | |
| | | Ethanolamine, | | | | | | |
| | | glucose | | | | | | |
| C | IMDM | Insulin, | 42.3 ± 6.7 | 95.4 ± 2.2 | 89 ± 11 | 87 ± 4 | 102 ± 5 | 100 ± 1 |
| | | Transferrin, | | | | | | |
| | | Selenium, | | | | | | |
| | | Ethanolamine, | | | | | | |
| | | glucose, 1.0% | | | | | | |
| | | HA | | | | | | |
| D | IMDM | Glucose, | 51.0 ± 13.3 | 94.7 ± 3.2 | 81 ± 0 | 83 ± 7 | 92 ± 4 | 89 ± 3 |
| | | 1.0% HA | | | | | | |
| E | IMDM | Ethanolamine, | 44.3 ± 5.9 | 93.4 ± 1.7 | 55 ± 1 | 57 ± 0 | 88 ± 5 | 94 ± 10 |
| | | Glucose | | | | | | |
| F | IMDM | Ethanolamine, | 44.0 ± 6.6 | 94.9 ± 2.4 | 90 ± 6 | 77 ± 1 | 102 ± 8 | 92 ± 2 |
| | | Glucose, | | | | | | |
| | | 1.0% HA | | | | | | |

As shown in Table 5, Groups C and F having identical media compositions except for the presence of insulin, transferrin, selenium, in Group C, showed good, and substantially the same, growth rate, viability, direct cytotoxicity and ADCC. In contrast, Group D, which has media compositions identical to Group F but for the absence of ethanolamine, had a cell growth rate of 16% slower than that of Group F (Group D showed a cell doubling time of 51 hours as compared to a doubling time of 44 hours in Group F). This indicates that ethanolamine is useful for maintaining desired growth rate. Group E, having a media composition that is identical to that of Group F but for the absence of HA, showed poor cytotoxicity, only 55% as compared to 90% at cycle 3, and only 57% as compared to 77% at cycle 5, indicating including HA in the customized NK-92® culture medium can boost cytotoxicity of the NK-92® cells.

### Example 3: Effect of HA Titration and Other Supplements: Insulin, Transferrin and Sodium Selenite (haNK® Cell Study)

Growth of haNK® cells in IMDM containing different supplements was compared with haNK® cells in reference growth medium.

HaNK® cells (NK-92® cells engineered to express CD16 and IL-2) is cultured in a reference growth medium comprising human AB serum and poloxamer 188. In this study, it was determined whether Iscove's Modified Dulbecos Medium (IMDM) supplemented with human AB serum, poloxamer 188 and additional supplements can support the growth, viability and functional activity of haNK® cells in a similar manner to haNK® reference growth medium. Previous studies demonstrated that an IMDM formulation supplemented with 10 mg/L insulin, 5.5 mg/L transferrin, 6.7 μg/L sodium selenite, 2.0 mg/L ethanolamine, 2.0 g/L glucose, and 1.0% human albumin supported growth and function of the aNK™ cell line. In these studies, ethanolamine and human albumin played a key role in supporting the growth and/or functional activity of aNK™. Accordingly, the present study was conducted to determine whether the optimal IMDM-based formulation would support the growth and function of haNK® cells.

The growth, viability and function of the haNK® cell line cultured in IMDM basal media containing human AB serum, poloxamer 188 and additional supplements (Table 6) was compared with haNK® reference growth medium. Since a key objective of this experiment was to determine the role of human albumin in supporting cell function, the concentration of this reagent was sequentially increased from 0.125% to 1.0% in groups C to F. haNK® cells were cultured in each media formulation for five growth cycles of 3 to 4 days in T75 tissue culture flasks. For each growth cycle, the cells were grown from ˜0.3×10⁶ cells/mL to ˜0.8-1.2×10⁶ cells/mL per growth cycle. Growth rate and viability was determined by automated cell counting at the time of seeding and harvest. Functional activity was measured as antibody-dependent cellular cytotoxicity (ADCC) against the Ramos cell line and direct cytotoxicity against K562 cells using Calcein AM release assays following standard methods at the end of the third and fifth growth cycles.

TABLE 6
Media Formulations

| Grp | Medium¹ | Additional Supplements | Abbreviation |
|---|---|---|---|
| A | Reference | None | None |
| | growth | | |
| | medium | | |
| B | IMDM | 10.0 mg/L Insulin, 5.5 mg/L Transferrin, 6.7 ug/L | Insulin, Transferrin, |
| | | Sodium Selenite², 2.0 mg/L Ethanolamine, 2.0 g/L | Selenium, |
| | | Glucose³ | Ethanolamine, |
| | | | Glucose |
| C | IMDM | 10.0 mg/L Insulin, 5.5 mg/L Transferrin, 6.7 ug/L | Insulin, Transferrin, |
| | | Sodium Selenite², 2.0 mg/L Ethanolamine, 2.0 g/L | Selenium, |
| | | Glucose³, 0.125% Human Albumin | Ethanolamine, |
| | | | Glucose, 0.125% HA |
| D | IMDM | 10.0 mg/L Insulin, 5.5 mg/L Transferrin, 6.7 ug/L | Insulin, Transferrin, |
| | | Sodium Selenite², 2.0 mg/L Ethanolamine, 2.0 g/L | Selenium, |
| | | Glucose³, 0.25% Human Albumin | Ethanolamine, |
| | | | Glucose, 0.25% HA |
| E | IMDM | 10.0 mg/L Insulin, 5.5 mg/L Transferrin, 6.7 ug/L | Insulin, Transferrin, |
| | | Sodium Selenite², 2.0 mg/L Ethanolamine, 2.0 g/L | Selenium, |
| | | Glucose³, 0.5% Human Albumin | Ethanolamine, |
| | | | Glucose, 0.5% HA |
| F | IMDM | 10.0 mg/L Insulin, 5.5 mg/L Transferrin, 6.7 ug/L | Insulin, Transferrin, |
| | | Sodium Selenite², 2.0 mg/L Ethanolamine, 2.0 g/L | Selenium, |
| | | Glucose³, 1.0% Human Albumin | Ethanolamine, |
| | | | Glucose, 1.0% HA |
| G | IMDM | 2.0 g/L Glucose³, 1.0% Human Albumin | Glucose, 1.0% HA |
| H | IMDM | 2.0 mg/L Ethanolamine, 2.0 g/L Glucose³ | EA, Glucose |
| I | IMDM | 2.0 mg/L Ethanolamine, 2.0 g/L Glucose³, 1.0% | Insulin, Transferrin, |
| | | Human Albumin | Selenium, |
| | | | Ethanolamine, |
| | | | Glucose, 1.0% HA |

¹All media were supplemented with Human AB Serum and Poloxamer 188.
²Sodium Selenite is present in the IMDM basal media formulation at 17 μg/L. The media was supplemented with 6.7 ug/L Sodium Selenite to give a final concentration of 23.7 μg/L Sodium Selenite.
³Glucose is present in the IMDM basal media formulation at 4.5 g/L. The media was supplemented with 2 g/L glucose to give a final concentration of 6.5 g/L glucose.

In this study, both basal media were supplemented with human AB serum and poloxamer 188. Sodium selenite is present in the IMDM basal media formulation at 17 μg/L. The media was supplemented with 6.7 μg/L sodium selenite to give a final concentration of 23.7 μg/L sodium selenite. Glucose is present in the IMDM basal media formulation at 4.5 g/L. The media was supplemented with 2 g/L glucose to give a final concentration of 6.5 g/L glucose.

TABLE 7
Study Results

| | | | | | Average ± SD % | | | |
|---|---|---|---|---|---|---|---|---|
| | | | Average ± SD | | Cytotoxicity⁴ | | Average ± SD % | |
| | | | Doubling | | against K562 at | | ADCC⁴ against | |
| | | Additional | Time | Average ± SD | E:T 10 | | Ramos at E:T 10 | |
| Grp. | Medium¹ | Supplements | (hours)² | Viability (%)³ | Cycle 3 | Cycle 5 | Cycle 3 | Cycle 5 |
| A | Reference | — | 36.2 ± 1.4 | 94.9 ± 2.6 | 72 ± 3 | 63 ± 3 | 97 ± 6 | 87 ± 1 |
| | growth | | | | | | | |
| | medium | | | | | | | |
| B | IMDM | Insulin, | 38.8 ± 5.8 | 93.9 ± 1.9 | 59 ± 4 | 57 ± 3 | 95 ± 11 | 94 ± 4 |
| | | Transferrin, | | | | | | |
| | | Selenium, | | | | | | |
| | | Ethanolamine, | | | | | | |
| | | glucose | | | | | | |
| C | IMDM | Insulin, | 35.8 ± 4.5 | 94.6 ± 2.7 | 73 ± 9 | 59 ± 1 | 102 ± 9 | 92 ± 6 |
| | | Transferrin, | | | | | | |
| | | Selenium, | | | | | | |
| | | Ethanolamine, | | | | | | |
| | | glucose, | | | | | | |
| | | 0.125% ha | | | | | | |
| D | IMDM | Insulin, | 40.2 ± 7.3 | 95.5 ± 2.0 | 78 ± 6 | 63 ± 6 | 103 ± 3 | 90 ± 4 |
| | | Transferrin, | | | | | | |
| | | Selenium, | | | | | | |
| | | Ethanolamine, | | | | | | |
| | | Glucose, | | | | | | |
| | | 0.25% HA | | | | | | |
| E | IMDM | Insulin, | 39.4 ± 7.9 | 95.8 ± 2.2 | 78 ± 2 | 71 ± 3 | 106 ± 2 | 94 ± 6 |
| | | Transferrin, | | | | | | |
| | | Selenium, | | | | | | |
| | | Ethanolamine, | | | | | | |
| | | Glucose, | | | | | | |
| | | 0.5% HA | | | | | | |
| F | IMDM | Insulin, | 41.3 ± 8.1 | 95.4 ± 2.2 | 89 ± 11 | 87 ± 4 | 102 ± 5 | 100 ± 1 |
| | | Transferrin, | | | | | | |
| | | Selenium, | | | | | | |
| | | Ethanolamine, | | | | | | |
| | | Glucose, | | | | | | |
| | | 1.0% HA | | | | | | |
| G | IMDM | Glucose, | 45.3 ± 8.8 | 95.1 ± 2.9 | 81 ± 0 | 83 ± 7 | 92 ± 4 | 89 ± 3 |
| | | 1.0% HA | | | | | | |
| H | IMDM | EA, | 43.1 ± 7.0 | 94.2 ± 1.8 | 55 ± 1 | 57 ± 0 | 88 ± 10 | 94 ± 10 |
| | | Glucose | | | | | | |
| I | IMDM | EA, | 41.0 ± 3.7 | 95.5 ± 2.3 | 90 ± 6 | 77 ± 1 | 102 ± 8 | 92 ± 2 |
| | | Glucose, | | | | | | |
| | | 1.0% HA | | | | | | |

¹All media were supplemented with Human AB Serum and Poloxamer 188 in addition to the supplements shown in the table.
²Average ± standard deviation doubling time was calculated for five growth cycles from viable cell density (VCD) of the aNK ™ cell culture determined at the start and end of each growth cycle using an NC200 automated cell counter using the following equation: Ln2/(Ln (VCD End of Cycle/VCD Start of Cycle)/Length of Cycle)).
³Average ± standard deviation viability was calculated for five growth cycles from the cell viability of the aNK ™ cell cultures determined at the start and end of each growth cycle using an NC200 automated cell counter.
⁴ADCC against Ramos cells and direct cytotoxicity against K562 cells were determined using Calcein AM release assays following standard methods.

In this study, all basal media were supplemented with human AB serum and poloxamer 188 in addition to the supplements shown in the table. Average±standard deviation doubling time was calculated for five growth cycles from viable cell density (VCD) of the aNK™ cell culture determined at the start and end of each growth cycle using an NC200 automated cell counter using the following equation: Ln2/(Ln (VCD End of Cycle/VCD Start of Cycle)/Length of Cycle)). Average±standard deviation viability was calculated for five growth cycles from the cell viability of the aNK™ cell cultures determined at the start and end of each growth cycle using an NC200 automated cell counter. ADCC against Ramos cells and direct cytotoxicity against K562 cells were determined using Calcein AM release assays following standard methods.

The results show that haNK® cells exhibited a similar growth rate, viability and functional activity when grown in IMDM basal media containing 0.125% human albumin (Group C versus Group A) with haNK® cells grown in a reference growth medium. Although increasing concentrations of human albumin enhanced the functional activity of haNK® cells, the growth rate of the cell line decreased and was more variable (Groups D-G) with higher human albumin concentrations. In contrast, absence of human albumin (Group B) resulted in acceptable growth rate, but marginally inferior functional activity, indicating the need for inclusion of this reagent.

Absence of insulin, transferrin, sodium selenite and ethanolamine supplementation from the optimal IMDM formulation (Group G versus Group F), resulted in reduced functional activity and a marginal decrease in growth rate of haNK® cells, indicating that one or more of these components are required to support growth and functional activity. The growth rate and functional activity of haNK® cells was marginally restored by supplementation with ethanolamine (Group G versus Group I), suggesting that this compound should be included alongside human albumin in the IMDM formulation.

The results from this study indicate that IMDM basal media supplemented with 10 mg/L insulin. 5.5 mg/L transferrin, 6.7 μg/L sodium selenite, 2.0 mg/L ethanolamine, 2.0 g/L glucose, 0.125% human albumin supported an equivalent growth rate, viability, and functional activity of haNK® cells compared to haNK® cells grown in a reference growth medium. These data also show that supplementation with human albumin enhanced the functional activity of haNK® cells grown in the IMDM basal media formulation. However, higher concentrations of this reagent slowed cell growth. Further, the results provide evidence that ethanolamine is the key component in insulin, transferrin, selenium, ethanolamine, in supporting the growth rate and functional activity of haNK® in the IMDM basal media formulation.

### Example 4: Identifying Optimal Concentrations of Human Albumin and Ethanolamine (haNK® Cell Study)

The optimal concentrations of human albumin and ethanolamine for the growth and function of haNK® cells (NK-92® engineered to express CD16 and IL-2) in IMDM-basal medium were determined. The medium do not contain insulin, transferrin, or sodium selenite.

Previous studies have shown that a IMDM basal medium containing human AB serum and poloxamer 188 and additionally supplemented with 10 mg/L insulin, 5.5 mg/L transferrin, 6.7 μg/L sodium selenite, 2.0 mg/L ethanolamine, 2.0 g/L glucose, 0.125% human albumin will support the growth and functional activity of haNK® cells. Moreover, these studies indicated that human albumin and ethanolamine were potentially key components of this formulation in supporting haNK® cell growth and/or function. Therefore, the present study sought to determine the optimal concentration of these reagents within the IMDM-basal medium formulation in the absence of insulin, transferrin and sodium selenite supplementation.

The growth and viability of haNK® cells cultured in an IMDM-basal medium formulation supplemented with human AB serum, poloxamer 188 , 2.0 g/L glucose and a range of concentrations of ethanolamine and human albumin was tested in a multi-parameter format within a 24-well G-Rex plate (Table 8). Control groups included in the study included duplicate wells of the reference growth medium and IMDM-basal medium containing human AB serum and poloxamer 188 additionally supplemented with 10 mg/L insulin, 5.5 mg/L transferrin, 6.7 μg/L sodium selenite, 2.0 mg/L ethanolamine, 2.0 g/L glucose, 1.0% human albumin. The haNK® cell line was cultured in each medium formulation for five growth cycles of 3 to 4 days in the G-Rex plate. For each growth cycle, the cells were grown from ˜0.3×10⁶ cells/mL to ˜0.8-1.2×10⁶ cells/mL per growth cycle. Cell growth rate and viability was determined by automated cell counting using NC-200™.

After the fifth growth cycle, selected groups were transferred to T75 flasks and cultured for a single growth cycle and functional activity was measured as antibody-dependent cell cytotoxicity (ADCC) against the Ramos cell line and direct cytotoxicity against K562 cells using Calcein AM release assays.

TABLE 8
Medium Formulations

| | | Plate Column | | | | | |
|---|---|---|---|---|---|---|---|
| | | 1 | 2 | 3 | 4 | 5 | 6² |
| Plate | A | 200 mg/L EA | 20 mg/L EA | 2 mg/L EA | 0.2 mg/L EA | 0.0 mg/L EA | Reference |
| Row | | 0.50% HA | 0.50% HA | 0.50% HA | 0.50% HA | 0.50% HA | growth |
| | | | | | | | medium |
| | B | 200 mg/L EA | 20 mg/L EA | 2 mg/L EA | 0.2 mg/L EA | 0.0 mg/L EA | Reference |
| | | 0.25% HA | 0.25% HA | 0.25% HA | 0.25% HA | 0.25% HA | growth |
| | | | | | | | medium |
| | C | 200 mg/L EA | 20 mg/L EA | 2 mg/L EA | 0.2 mg/L EA | 0.0 mg/L EA | IMDM |
| | | 0.125% HA | 0.125% HA | 0.125% HA | 0.125% HA | 0.125% HA | |
| | D | 200 mg/L EA | 20 mg/L EA | 2 mg/L EA | 0.2 mg/L EA | 0.0 mg/L EA | IMDM |
| | | 0.00% HA | 0.00% HA | 0.00% HA | 0.00% HA | 0.00% HA | |

In this study, wells from columns 1 5 contained IMDM-basal medium supplemented with human AB serum, poloxamer 188, 2.0 g/L glucose and the specified concentrations of ethanolamine (EA) and human albumin (HA). Control wells A6 and B6 contained reference growth medium; Control wells C6 and D6 contained IMDM-basal medium supplemented with human AB serum, poloxamer 188, 10 mg/L insulin, 5.5 mg/L transferrin, 6.7 μg/L sodium selenite, 2.0 mg/L ethanolamine, 2.0 g/L glucose, and 1.0% human albumin.

The growth rate and viability of haNK® cells in the medium formulations are summarized in Table 9.

TABLE 9
haNK ® Growth Rate and Cell Viability

| Average ± SD | | | | | | | | |
|---|---|---|---|---|---|---|---|---|
| Doubling | | | | | | | | |
| Time (hours)¹ | | Ethanolamine³ | | | | | | |
| Average ± SD | | | 200 mg/L | 20 mg/L | 2 mg/L | 0.2 mg/L | 0.0 mg/L | Controls⁴ |
| Viability (%)² | | | 1 | 2 | 3 | 4 | 5 | 6 |
| HA³ | 0.50% | A | 51.43 ± 11.3 | 46.99 ± 7.7 | 48.54 ± 8.5 | 47.94 ± 6.6 | 44.63 ± 7.5 | 41.04 ± 3.9 |
| | | | 95.5 ± 0.8 | 96.0 ± 0.6 | 95.7 ± 0.7 | 95.4 ± 0.8 | 95.4 ± 0.8 | 96.1 ± 1.3 |
| | 0.25% | B | 50.24 ± 11.6 | 46.57 ± 7.0 | 48.38 ± 8.5 | 47.20 ± 7.7 | 47.34 ± 5.2 | 41.54 ± 3.0 |
| | | | 95.4 ± 1.1 | 95.9 ± 0.6 | 95.5 ± 0.6 | 95.5 ± 0.7 | 95.8 ± 1.2 | 96.2 ± 1.2 |
| | 0.125% | C | 52.37 ± 12.6 | 47.16 ± 9.7 | 47.51 ± 7.2 | 47.78 ± 8.4 | 47.73 ± 5.3 | 53.06 ± 9.8 |
| | | | 95.1 ± 1.1 | 95.5 ± 0.7 | 95.4 ± 0.8 | 95.1 ± 0.8 | 95.6 ± 1.3 | 94.9 ± 1.4 |
| | 0.0% | D | 58.89 ± 15.8 | 49.06 ± 11.5 | 48.39 ± 10.6 | 48.26 ± 9.7 | 46.74 ± 7.7 | 50.54 ± 6.6 |
| | | | 94.4 ± 1.0 | 95.0 ± 1.0 | 94.9 ± 0.9 | 94.8 ± 0.8 | 95.1 ± 1.6 | 95.4 ± 1.3 |

¹Average ± standard deviation doubling time was calculated for five growth cycles from viable cell density (VCD) of the haNK ® cell culture determined at the start and end of each growth cycle using an automated cell counter using the following equation: Ln2/(Ln (VCD End of Cycle/VCD Start of Cycle)/Length of Cycle)). The doubling time is shown in the top of each cell in the table.
²Average ± standard deviation viability was calculated for five growth cycles from the cell viability of the haNK ® cell cultures determined at the start and end of each growth cycle using an automated cell counter. The viability is shown in the bottom of each cell in the Table.
³Wells from columns 1-5 contained IMDM-basal media supplemented with Human AB Serum, Poloxamer 188, 2 g/L Glucose and the specified concentrations of Ethanolamine (EA) and Human Albumin (HA).
⁴Control wells A6 and B6 contained haNK ® reference growth medium; Control wells C6 and D6 contained IMDM-basal media supplemented with Human AB Serum, Poloxamer 188, 10 mg/L Insulin, 5.5 mg/L Transferrin, 6.7 μg/L Sodium Selenite, 2.0 mg/L Ethanolamine, 2.0 g/L Glucose, and 1.0% Human Albumin.

In this study, average ±standard deviation doubling time was calculated for five growth cycles from viable cell density (VCD) of the haNK® cell culture determined at the start and end of each growth cycle using an automated cell counter using the following equation: Ln2/(Ln (VCD End of Cycle/VCD Start of Cycle)/Length of Cycle)). The doubling time is shown in the top of each cell in Table 9.

Average±standard deviation viability was calculated for five growth cycles from the cell viability of the haNK® cell cultures determined at the start and end of each growth cycle using an automated cell counter. The viability is shown in the bottom of each cell in Table 9.

Wells from columns 1-5 contained IMDM-basal medium supplemented with human AB serum, poloxamer 188, 2 g/L glucose and the specified concentrations of ethanolamine (EA) and human albumin (HA). Control wells A6 and B6 contained reference growth medium; Control wells C6 and D6 contained IMDM-basal medium supplemented with human AB serum, poloxamer 188, 10 mg/L insulin, 5.5 mg/L transferrin, 6.7 μg/L sodium selenite, 2.0 mg/L ethanolamine, 2.0 g/L glucose, and 1.0% human albumin.

The doubling time and viability were similar between haNK® cells grown in IMDM-basal medium formulations that contained 0-20 mg/L of ethanolamine and 0.0%-0.5% human albumin. However, medium formulations that contained 200 mg/L of ethanolamine showed higher and more variable doubling times, indicating that at this concentration of ethanolamine was inhibiting the growth of the haNK® cells.

None of the test formulations showed superior growth rate or viability to the reference growth medium control, indicating that supplementation with combinations of ethanolamine and human albumin does not compensate for the absence of insulin, transferrin and sodium selenite supplementation in the IMDM-basal formulation.

The IMDM control formulation exhibited longer doubling times than the reference growth medium control and test formulations, which is consistent with previous observations that a higher concentration of human albumin inhibits haNK® cell growth.

For functional testing, selected groups were expanded in T75 flasks for one growth cycle and tested for direct cytotoxicity against K562 cells and for ADCC against Ramos Cells (Table 10).

TABLE 10
Functional Testing

| | | | Average ± SD % | Average ± SD % |
|---|---|---|---|---|
| | | | Cytotoxicity² | ADCC² |
| | | Additional | against | against |
| Grp. | Medium¹ | Supplements | K562 at E:T 10 | Ramos at E:T 10 |
| B2 | IMDM | 20 mg EA, | 52 ± 3 | 96 ± 14 |
| | | 0.25% HA | | |
| B3 | IMDM | 2 mg EA, | 58 ± 1 | 96 ± 9 |
| | | 0.25% HA | | |
| B5 | IMDM | 0 mg EA, | 56 ± 5 | 89 ± 4 |
| | | 0.25% HA | | |
| C2 | IMDM | 20 mg EA, | 59 ± 4 | 93 ± 6 |
| | | 0.125% HA | | |
| C3 | IMDM | 2 mg EA, | 61 ± 3 | 88 ± 7 |
| | | 0.125% HA | | |
| C4 | IMDM | 0 mg EA, | 48 ± 3 | 93 ± 8 |
| | | 0.125% HA | | |
| A6 | Reference | — | 66 ± 3 | 91 ± 8 |
| | growth | | | |
| | medium | | | |
| D5 | IMDM | Insulin, | 90 ± 6 | 81 ± 7 |
| | | Transferrin, | | |
| | | Selenium, | | |
| | | Ethanolamine, | | |
| | | 1.0% HA | | |

¹IMDM basal media were supplemented with Human AB Serum, Poloxamer 188, 2 g/L Glucose in addition to listed supplements.
²ADCC against Ramos cells and direct cytotoxicity against K562 cells were determined using Calcein AM release assays following standard methods.

In this study, basal medium were supplemented with human AB serum, poloxamer 188, 2.0 g/L glucose in addition to listed supplements. ADCC against Ramos cells and direct cytotoxicity against K562 cells were determined using Calcein AM release assays following standard methods.

The haNK® cell line cultured in IMDM-basal medium containing 0.25% human albumin with reducing concentrations of ethanolamine showed similar ADCC against Ramos cells and direct cytotoxicity against K562 target cells. In contrast, cells cultured in IMDM-basal medium containing 0.125% human albumin showed a drop in direct cytotoxicity against K562 at when ethanolamine was absent from the medium. Therefore, ethanolamine may support the functional activity of haNK® when human albumin is lowered in the IMDM basal formulation in the absence of insulin, transferrin and sodium selenite supplementation.

The results indicate that supplementation of the IMDM basal-medium (containing human AB serum and poloxamer 188) with 0.2-20 mg/L ethanolamine and 0.125%-0.5% HA did not alter the viability of haNK® cells. Ethanolamine at 2 or 20 mg/L enhanced the direct cytotoxicity of haNK® cells in IMDM-basal medium supplemented with 0.125% human albumin.

The growth rate and functional activity of haNK® cells in the IMDM-basal formulations tested in this study was lower than that of the reference growth medium control, indicating that the presence of insulin, transferrin and sodium selenite supplementation is useful to maintain the functional activity and growth rate of this cell line.

### Example 5: Evaluation of the Growth of haNK® Cells With and Without Ethanolamine Supplementation

Growth of haNK® cells was evaluated in the current formulation of IMDM-basal medium with and without ethanolamine supplementation, as well as compared with the growth and function of haNK® cells in reference growth medium.

As shown in Example 3, a IMDM basal medium containing human AB serum and poloxamer 188, additionally supplemented with 10 mg/L insulin, 5.5 mg/L transferrin, 6.7 μg/L sodium selenite, 2.0 mg/L ethanolamine, 2.0 g/L glucose, 0.125% human albumin will support growth and functional activity of haNK® cells. As shown in Example 4, ethanolamine enhanced the functional activity of haNK® cells in the presence of 0.125% human albumin in IMDM basal medium supplemented with human AB serum, poloxamer 188 and 2.0 g/L glucose. Accordingly, the present study sought to confirm that the IMDM-based formulation containing human AB serum and poloxamer 188 and additionally supplemented with 10 mg/L insulin, 5.5 mg/L transferrin, 6.7 μg/L sodium selenite, 2.0 g/L glucose, 0.125% human albumin would support the growth and function of haNK® cells with and without additional supplementation of 2.0 mg/L of ethanolamine.

The growth, viability and function of haNK® cells cultured in IMDM basal medium containing human AB serum, poloxamer 188 and additional supplements (Table 8) was compared with haNK® cells cultured in haNK® reference growth medium. Since a key objective of this experiment was to determine the role of ethanolamine in supporting cell growth and function, this reagent was excluded from the medium formulation used in Group C (Table 11). haNK® cells were cultured in each medium formulation for five growth cycles of 3 to 4 days in T75 tissue culture flasks. For each growth cycle, the cells were grown from ˜0.3×10⁶ cells/mL to ˜0.8-1.2×10⁶ cells/mL per growth cycle. Growth rate and viability was determined by automated cell counting at the time of seeding and harvest. Functional activity was measured as antibody-dependent cellular cytotoxicity (ADCC) against the Ramos cell line and direct cytotoxicity against K562 cells using Calcein AM release assays following standard methods at the end of the third and fifth growth cycles.

TABLE 11
Medium Formulations

| Grp | Medium¹ | Additional Supplements | Abbreviation |
|---|---|---|---|
| A | Reference | None | None |
| | growth | | |
| | medium | | |
| B | IMDM | 10.0 mg/L Insulin, 5.5 mg/L Transferrin, 6.7 μ/L | Insulin, Transferrin, |
| | | Sodium Selenite², 2.0 g/L Glucose³, 0.125% Human | Selenium- |
| | | Albumin, 2.0 mg/L Ethanolamine | Ethanolamine, |
| | | | Glucose, 0.125% HA |
| C | IMDM | 10.0 mg/L Insulin, 5.5 mg/L Transferrin, 6.7 μg/L | Insulin, Transferrin, |
| | | Sodium Selenite², 2.0 g/L Glucose³, 0.125% Human | Selenium, Glucose, |
| | | Albumin | 0.125% HA |

¹Both basal media were supplemented with Human AB Serum and Poloxamer 188.
²Sodium Selenite is present in the IMDM basal media formulation at 17 μg/L. The media was supplemented with 6.7 μg/L Sodium Selenite to give a final concentration of 23.7 μg/L Sodium Selenite.
³Glucose is present in the IMDM basal media formulation at 4.5 g/L. The media was supplemented with 2 g/L glucose to give a final concentration of 6.5 g/L glucose.

In this study, both basal medium were supplemented with human AB serum and poloxamer 188. Sodium selenite is present in the IMDM basal medium formulation at 17 μg/L. The medium was supplemented with 6.7 μg/L sodium selenite to give a final concentration of 23.7 μg/L sodium selenite. Glucose is present in the IMDM basal medium formulation at 4.5 g/L. The medium was supplemented with 2.0 g/L glucose to give a final concentration of 6.5 g/L glucose.

The results of the study are summarized in Table 12.

TABLE 12
Study Results

| | | | | | Average ± SD % | | | |
|---|---|---|---|---|---|---|---|---|
| | | | Average ± SD | | Cytotoxicity⁴ | | Average ± SD % | |
| | | | Doubling | Average ± SD | against K562 at | | ADCC⁴ against | |
| | | Additional | Time | Viability | E:T 10 | | Ramos at E:T 10 | |
| Grp. | Medium¹ | Supplements | (hours)² | (%)³ | Cycle 3 | Cycle 5 | Cycle 3 | Cycle 5 |
| A | Reference | — | 34.3 ± 3.1 | 96.8 ± 0.9 | 66 ± 6 | 70 ± 4 | 97 ± 6 | 108 ± 10 |
| | growth | | | | | | | |
| | medium | | | | | | | |
| B | IMDM | Insulin, | 36.5 ± 3.1 | 96.3 ± 1.4 | 53 ± 1 | 78 ± 4 | 98 ± 9 | 116 ± 14 |
| | | Transferrin, | | | | | | |
| | | Selenium- | | | | | | |
| | | Ethanolamine, | | | | | | |
| | | Glucose, | | | | | | |
| | | 0.125% HA | | | | | | |
| C | IMDM | Insulin, | 37.6 ± 3.3 | 96.1 ± 1.4 | 46 ± 6 | 51 ± 5 | 102 ± 8 | 99 ± 7 |
| | | Transferrin, | | | | | | |
| | | Selenium, | | | | | | |
| | | Glucose, | | | | | | |
| | | 0.125% HA | | | | | | |

¹All basal media were supplemented with Human AB Serum and Poloxamer 188 in addition to the supplements shown in the table.
²Average ± standard deviation doubling time was calculated for five growth cycles from viable cell density (VCD) of the haNK ® cell culture determined at the start and end of each growth cycle using an NC200 automated cell counter using the following equation: Ln2/(Ln (VCD End of Cycle/VCD Start of
Cycle)/Length of Cycle)).
³Average ± standard deviation viability was calculated for five growth cycles from the cell viability of the aNK ™ cell cultures determined at the start and end of each growth cycle using an NC200 automated cell counter.
⁴ADCC against Ramos cells and direct cytotoxicity against K562 cells were determined using Calcein AM release assays following standard methods.

All basal medium were supplemented with human AB serum and poloxamer 188 in addition to the supplements shown in the table.

Average±standard deviation doubling time was calculated for five growth cycles from viable cell density (VCD) of the haNK® cell culture determined at the start and end of each growth cycle using an NC-200™ automated cell counter using the following equation: Ln2/(Ln (VCD End of Cycle/VCD Start of Cycle)/Length of Cycle)).

Average±standard deviation viability was calculated for five growth cycles from the cell viability of the haNK® cell cultures determined at the start and end of each growth cycle using an NC-200™ automated cell counter.

ADCC against Ramos cells and direct cytotoxicity against K562 cells were determined using Calcein AM release assays following standard methods.

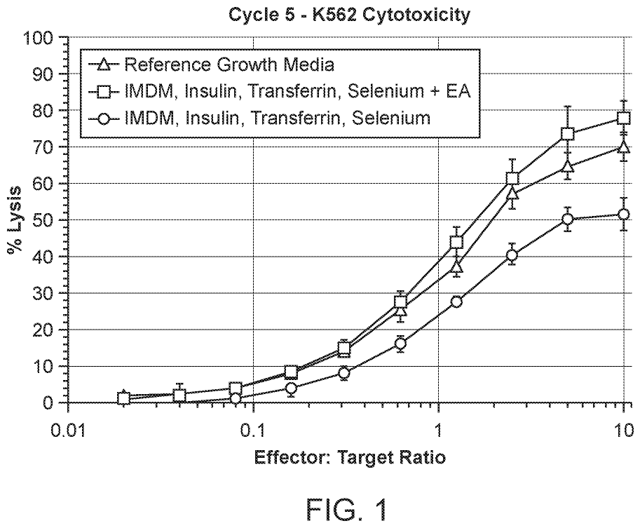

HaNK® cells exhibited a similar growth rate and viability to haNK® cells grown in reference growth medium when grown in IMDM basal medium containing supplements with (Group B versus Group A) and without (Group C versus Group A) ethanolamine. However, the direct cytotoxic function of the haNK® cell line was marginally impaired at Cycle 3 and more profoundly impaired at Cycle 5 in the absence of ethanolamine supplementation (Table 12 and FIG. 1), indicating ethanolamine is important to maintain the functional activity in the current IMDM based medium formulation. As shown in FIG. 1, haNK® cells were grown for five growth cycles in the indicated medium and were assessed for their direct cytotoxic activity against K562 cells in a Calcein AM-based cytotoxicity assay at the indicated effector to target ratios. Direct cytotoxicity function of the haNK® cell line in medium supplemented with ethanolamine (“IMDM+Insulin, Transferrin, Selenium, Ethanolamine”) was significantly higher than the medium that is not supplemented (“IMDM+Insulin, Transferrin, Selenium”).

The results show that IMDM basal medium containing human AB serum and poloxamer 188 and additionally supplemented with 10 mg/L insulin, 5.5 mg/L transferrin, 6.7 μg/L sodium selenite, 2.0 g/L glucose, 0.125% human albumin and 2.0 mg/L ethanolamine supports an equivalent growth rate, viability, and functional activity of haNK® cells compared to haNK® cells grown in haNK® reference growth medium. The results also indicate that supplementation with ethanolamine enhances the functional activity of haNK® cells grown in the IMDM basal medium formulation.

### Example 6: Phenotypes (haNK® Cell Study)

haNK® cells grown in the medium compositions as described in Table 13 were assayed for the surface expression of various markers for NK-92® cells, CD56, CD3, CD54, CD16, NKG2D, and NKp30 by flow cytometry. The percentages of cell surface marker expression of the markers are shown in Table 13.

TABLE 13
Expression of various surface markers

| | | Ave % Cell Surface Marker Expression | | | | | |
|---|---|---|---|---|---|---|---|
| Grp. | Growth Medium | CD56 | CD3 | CD54 | CD16 | NKG2D | NKp30 |
| | Target Range | >90% | <5% | >85% | >90% | >80% | >80% |
| | haNK ® Ref* | 99.28 | 0.13 | 99.46 | 94.88 | 90.92 | 98.55 |
| A | Reference growth medium | 99.54 | 0.31 | 99.44 | 96.89 | 94.76 | 98.74 |
| B | IMDM + Insulin, Transferrin, | 98.58 | 0.15 | 95.58 | 95.63 | 96.03 | 98.38 |
| | Selenium, Ethanolamine, | | | | | | |
| | Glucose | | | | | | |
| C | IMDM + Insulin, Transferrin, | 98.91 | 0.19 | 95.57 | 96.54 | 95.81 | 98.21 |
| | Selenium, Ethanolamine, | | | | | | |
| | Glucose, 0.125% HA | | | | | | |
| D | IMDM + Insulin, Transferrin, | 98.96 | 0.22 | 98.49 | 95.69 | 95.83 | 98.11 |
| | Selenium, Ethanolamine, | | | | | | |
| | Glucose, 0.25% HA | | | | | | |
| E | IMDM + Insulin, Transferrin, | 98.81 | 0.09 | 99.25 | 97.05 | 95.40 | 98.45 |
| | Selenium, Ethanol amine, | | | | | | |
| | Glucose, 1.0% HA | | | | | | |
| F | IMDM, Glucose, 1.0% HA | 97.80 | 0.15 | 98.45 | 95.71 | 96.48 | 98.41 |
| G | IMDM, Glucose, EA | 97.69 | 0.04 | 99.20 | 96.04 | 95.15 | 98.01 |
| H | IMDM, Glucose, EA, 1.0% | 97.07 | 0.14 | 97.09 | 95.78 | 95.62 | 98.00 |
| | HA | | | | | | |

*Note that haNK ® Ref. are haNK ® cells that were grown in refrence growth media containing human AB serum and Poloxamer 188.

The results show that cells in Groups C-F all had expression of the markers within the target range, indicating that the NK-92® culture medium as disclosed herein does not affect NK-92® cell phenotype.

### Example 7: Large Scale Expansion of haNK® Cells in NK-92® Culture Media

To determine media formulation robustness and scalability, haNK® cells were grown in the NK-92® culture media, described in Table 14 and 15, at large scale culture volumes equal to and greater than 5 liters in a bioreactor. The cells were grown from ˜0.3×10⁶ cells/mL to ˜0.8-1.2×10⁶ cells/mL per growth cycle and growth rate (doubling time) and viability was determined by automated cell counting at the time of seeding and harvest. Functional activity was measured as antibody dependent cellular cytotoxicity (ADCC) against Ramos cells in the presence of 1 μg Rituxan using Calcein AM release assays following standard methods during, at the of production and following cyropresevation and thaw. The surface expression of various markers for NK-92 cells, CD56, CD3, CD54, CD16, NKG2D, NKp30 was measured using flow cytometry. The percentages of cell surface marker expression of the markers are shown in Table 15 below.

TABLE 14
haNK ® Cell Scale-Up: Growth, Viability and Cytotoxic Function

| | | | Average ± SD | | |
|---|---|---|---|---|---|
| | | | Doubling Time | Average | Average ± SD |
| Medium¹ | Additional Supplements | Process Stage | (hours)² | Viability (%)³ | % ADCC⁴ |
| NK-92 ® | Insulin, Transferrin, | In Process | 37.0 ± 3.7 | 91.0 | 98.0 ± 3.0 |
| Culture | Selenium, Ethanolamine, | Testing | | | |
| Media | Glucose, HA | Bulk Drug | NA | 94.1 | 100.0 ± 1.0 |
| | | Substance | | | |
| | | Post Thaw | NA | 92.9 | 91.0 ± 4.0 |

¹NK-92 ® Culture media were supplemented with 5% Human AB Serum and 0.05% Poloxamer 188 in addition to the supplement formula shown in the table.
²Average ± standard deviation doubling time was calculated across each growth cycle from viable cell density (VCD) of the cell culture determined at the start and end of each growth cycle using an NC200 automated cell counter using the following equation: Ln2/(Ln (VCD End of Cycle/VCD Start of Cycle)/Length of Cycle)).
³Average viability was measured at the specified process stage using an NC200 automated cell counter.
⁴Average ± standard deviation % ADCC was measured at the specified process stage for an effector:target (E:T) ratio of 10 against Ramos target cells, in the presence of 1 μg Rituxan, using a Calcein AM release assay following standard methods.

TABLE 15
haNK ® Cell Scale-Up: Immune Phenotype Characteristics

| | | Average % Cell Surface Marker Expression | | | | | |
|---|---|---|---|---|---|---|---|
| Growth Medium¹ | Process Stage | CD56 | CD3 | CD54 | CD16 | NKG2D | NKp30 |
| Target Range - - - → | | >90% | <5% | >85% | >90% | >80% | >80% |
| Customized NK-92 ® | In Process | 99.6 | 0.06 | 99.8 | 96.9 | 88.7 | 97.7 |
| Media | Testing | | | | | | |
| | Bulk Drug | 99.6 | 0.03 | 99.8 | 96.5 | 86.5 | 96.6 |
| | Substance | | | | | | |
| | Post Thaw | 99.2 | 0.03 | 99.4 | 97.0 | 81.6 | 97.7 |

¹Customized NK-92 ® basal media were supplemented with 5% Human AB Serum and 0.05% Poloxamer 188 in addition to the supplements shown in the table.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

INFORMAL SEQUENCE LISTING

SEQ ID NO: 1 High Affinity Variant Immunoglobulin Gamma Fc Region Receptor III-A
nucleic acid sequence (full length form).
ATGTGGCA GCTGCTGCTG CCTACAGCTC TCCTGCTGCT GGTGTCCGCC
GGCATGAGAA CCGAGGATCT GCCTAAGGCC GTGGTGTTCC TGGAACCCCA
GTGGTACAGA GTGCTGGAAA AGGACAGCGT GACCCTGAAG TGCCAGGGCG
CCTACAGCCC CGAGGACAAT AGCACCCAGT GGTTCCACAA CGAGAGCCTG
ATCAGCAGCC AGGCCAGCAG CTACTTCATCGACGCCGCCA CCGTGGACGA
CAGCGGCGAG TATAGATGCC AGACCAACCT GAGCACCCTGAGCGACCCCG
TGCAGCTGGA AGTGCACATC GGATGGCTGC TGCTGCAGGC
CCCCAGATGGGTGTTCAAAG AAGAGGACCC CATCCACCTG AGATGCCACT
CTTGGAAGAA CACCGCCCTGCACAAAGTGA CCTACCTGCA GAACGGCAAG
GGCAGAAAGT ACTTCCACCA CAACAGCGAC TTCTACATCC CCAAGGCCAC
CCTGAAGGAC TCCGGCTCCT ACTTCTGCAG AGGCCTCGTGGGCAGCAAGA
ACGTGTCCAG CGAGACAGTG AACATCACCA TCACCCAGGG
CCTGGCCGTGTCTACCATCA GCAGCTTTTT CCCACCCGGC TACCAGGTGT
CCTTCTGCCT CGTGATGGTGCTGCTGTTCG CCGTGGACAC CGGCCTGTAC
TTCAGCGTGA AAACAAACAT CAGAAGCAGCACCCGGGACT GGAAGGACCA
CAAGTTCAAG TGGCGGAAGG ACCCCCAGGA CAAGTGA
SEQ ID NO: 2 High Affinity Variant Immunoglobulin Gamma Fc Region Receptor III-A
amino acid sequence (full length form). The Val at position 176 is underlined.
Met Trp Gln Leu Leu Leu Pro Thr Ala Leu Leu Leu Leu Val Ser Ala Gly Met Arg Thr Glu Asp
Leu Pro Lys Ala Val Val Phe Leu Glu Pro Gln Trp Tyr Arg Val Leu Glu Lys Asp Ser Val Thr
Leu Lys Cys Gln Gly Ala Tyr Ser Pro Glu Asp Asn Ser Thr Gln Trp Phe His Asn Glu Ser Leu
Ile Ser Ser Gln Ala Ser Ser Tyr Phe Ile Asp Ala Ala Thr Val Asp Asp Ser Gly Glu Tyr Arg Cys
Gln Thr Asn Leu Ser Thr Leu Ser Asp Pro Val Gln Leu Glu Val His Ile Gly Trp Leu Leu Leu
Gln Ala Pro Arg Trp Val Phe Lys Glu Glu Asp Pro Ile His Leu Arg Cys His Ser Trp Lys Asn
Thr Ala Leu His Lys Val Thr Tyr Leu Gln Asn Gly Lys Gly Arg Lys Tyr Phe His His Asn Ser
Asp Phe Tyr Ile Pro Lys Ala Thr Leu Lys Asp Ser Gly Ser Tyr Phe Cys Arg Gly Leu Val Gly
Ser Lys Asn Val Ser Ser Glu Thr Val Asn Ile Thr Ile Thr Gln Gly Leu Ala Val Ser Thr Ile Ser
Ser Phe Phe Pro Pro Gly Tyr Gln Val Ser Phe Cys Leu Val Met Val Leu Leu Phe Ala Val Asp
Thr Gly Leu Tyr Phe Ser Val Lys Thr Asn Ile Arg Ser Ser Thr Arg Asp Trp Lys Asp His Lys
Phe Lys Trp Arg Lys Asp Pro Gln Asp Lys
SEQ ID NO: 3 ER IL-2 nucleic acid sequence
ATGTACCGGATG CAGCTGCTGA GCTGTATCGC CCTGTCTCTG GCCCTCGTGA
CCAACAGCGC CCCTACCAGC AGCAGCACCA AGAAAACCCA GCTGCACCTG
GAACATCTGC TGCTGGACCTGCAGATGATC CTGAACGGCA TCAACAACTA
CAAGAACCCC AAGCTGACCC GGATGCTGACCTTCAAGTTC TACATGCCCA
AGAAGGCCAC CGAACTGAAA CATCTGCAGT GCCTGGAAGAGGAACTGAAG
CCCCTGGAAG AAGTGCTGAA CCTGGCCCAG AGCAAGAACT
TCCACCTGAGGCCCAGGGAC CTGATCAGCA ACATCAACGT GATCGTGCTG
GAACTGAAAG GCAGCGAGACAACCTTCATG TGCGAGTACG CCGACGAGAC
AGCTACCATC GTGGAATTTC TGAACCGGTGGATCACCTTC TGCCAGAGCA
TCATCAGCAC CCTGACCGGC TCCGAGAAGG ACGAGCTGTGA
SEQ ID NO: 4 ER IL-2 (ER retention signal is underlined) amino acid sequence
Met Tyr Arg Met Gln Leu Leu Ser Cys Ile Ala Leu Ser Leu Ala Leu Val Thr Asn Ser Ala Pro
Thr Ser Ser Ser Thr Lys Lys The Gln Leu Gln Leu Glu His Leu Leu Leu Asp Leu Gln Met Ile
Leu Asn Gly Ile Asn Asn Tyr Lys Asn Pro Lys Leu Thr Arg Met Leu Thr Phe Lys Phe Tyr Met
Pro Lys Lys Ala Thr Glu Leu Lys His Leu Gln Cys Leu Glu Glu Glu Leu Lys Pro Leu Glu Glu
Val Leu Asn Leu Ala Gln Ser Lys Asn Phe His Leu Arg Pro Arg Asp Leu Ile Ser Asn Ile Asn
Val Ile Val Leu Glu Leu Lys Gly Ser Glu The The Phe Met Cys Glu Tyr Ala Asp Glu Thr Ala
Thr Ile Val Glu Phe Leu Asn Arg Trp Ile Thr Phe Cys Gln Ser Ile Ile Ser Thr Leu The Gly Ser
Glu Lys Asp Glu Leu

What is claimed is:

1. A method of culturing NK-92® cells comprising culturing NK-92® cells in a customized NK-92® culture medium comprising a basal medium and one or more supplements,
wherein the one or more supplements comprise ethanolamine, an ethanolamine derivative, insulin, transferrin, sodium selenite, HA, or a combination thereof; and
wherein the basal medium comprises inorganic salts, vitamins and amino acids.

2. The method of claim 1, wherein the ethanolamine derivative is an ethanolamide.

3. The method of claim 2, wherein the ethanolamide is vaccenic acid ethanolamide, or oleic acid ethanolamide, palmitic acid ethanolamide, or stearic acid ethanolamide,

4. The method of claim 1, wherein the ethanolamine derivative is phosphatidylethanolamine.

5. The method of any of claims 1-4, wherein the one or more supplements comprise 1-7% human AB serum.

6. The method of any of claims 1-5, wherein the one or more supplements further comprise insulin, transferrin, selenium, or a combination thereof.

7. The method of any of claims 1-6, wherein the one or more supplements further comprise 300-600 IU/mL interleukin-2.

8. The method of any of claims 1-7, wherein the one or more supplements further comprise 0.01% to 0.1% of poloxamer 188.

9. The method of any of claims 1-8, wherein the NK-92® cells cultured in the customized NK-92® culture medium have substantially the same or higher cytotoxicity, growth rate, and viability compared to NK-92® cells grown in a reference growth medium.

10. The method of any of claims 1-9, wherein the NK-92® cells cultured in the customized NK-92® culture medium have 85-100% viability.

11. The method of any of claims 1-10, wherein the NK-92® cells cultured in the customized NK-92® culture medium show a direct cytotoxicity of and/or an ADCC of 60-100% at an effector to target ratio of 10:1.

12. The method of any of claims 1-11, wherein the NK-92® cells cultured in the customized NK-92® culture medium have a doubling time of 30-50 hours.

13. The method of any of claims 1-12, wherein the NK-92® cells express a cytokine, Fc Receptor, a chimeric antigen receptor, or a combination thereof.

14. The method of claims 1-13, wherein the customized NK-92® culture medium comprises 0.05-40 mg/L of ethanolamine, an ethanolamine derivative, or a combination thereof.

15. The method of claims 1-14, wherein the customized NK-92® culture medium 4.5-20 g/L of glucose.

16. The method of any of claims 1-15, wherein the customized NK-92® culture medium is further supplemented with 0.05% to 1.0% HA.

17. A cell culture comprising NK-92® cells in a customized NK-92® culture medium comprising a basal medium and one or more supplements,
wherein the one or more supplements comprise ethanolamine, an ethanolamine derivative, insulin, transferrin, sodium selenite, HA, or a combination thereof; and
wherein the basal medium comprises inorganic salts, vitamins and amino acids.

18. A cell culture comprising NK-92® cells in a customized NK-92® culture medium comprising a basal medium and one or more supplements,
wherein the one or more supplements comprise ethanolamine, an ethanolamine derivative, or a combination thereof; and
wherein the basal medium comprises inorganic salts, vitamins and amino acids.

19. The cell culture of claim 17 or 18, wherein the ethanolamine derivative is ethanolamide.

20. The cell culture of claim 19, wherein the ethanolamide is cis-vaccenic acid ethanolamide or oleic acid ethanolamide.

21. The cell culture of claim 18, wherein one or more supplements further comprise insulin, transferrin, selenium, or a combination thereof.

22. The cell culture of any of claims 17-21, wherein the one or more supplements further comprise 0.01% to 0.1% of poloxamer 188.

23. The cell culture of any of claims 17-22, wherein the customized NK-92® culture medium comprises 4.5-20 g/L glucose.

24. The cell culture of any of claims 17-23, wherein the one or more supplements comprise 0.05% to 1.0% HA.

25. The cell culture of any of claims 17-24, wherein the NK-92® cells express a cytokine, Fc Receptor, a chimeric antigen receptor, or a combination thereof.

26. The cell culture of any of claims 17-25, wherein the customized NK-92® culture medium comprises insulin, transferrin, selenium, or a combination thereof.

27. The cell culture of any of claims 17-26, wherein the NK-92® cells have substantially the same or higher cytotoxicity, growth rate, and/or viability as compared to control NK-92® cells.

28. The cell culture of any of claims 17-27, wherein the NK-92® cells that have been cultured in the customized NK-92® culture medium have 85-100% viability.

29. The cell culture of any of claims 17-28, wherein the NK-92® cells that have been cultured in the customized NK-92® culture medium the doubling time of the NK-92® cells is 30-50 hours.

30. The cell culture of any of claims 17-29, wherein the NK-92® cells show a direct cytotoxicity and/or an ADCC of 60-100% when using an effector: target ratio of 10:1.

31. The method of any of the claims 1-16, wherein the volume of the customized NK-92® culture medium is at least 5 liters.

32. The cell culture of any of the claims 17-30, wherein the cell culture volume is at least 5 liters.

33. The cell culture of any of the claims 17-30, wherein the NK-92® cells maintain substantially the same viability and/or cytotoxicity after the cells are crypreserved and thawed.


SEQUENCE LISTING (4 sequences)

SEQ ID NO: 1 - DNA, 765 bp, Artificial Sequence
  source: /note=“Description of Artificial Sequence Synthetic polynucleotide”
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 2 - protein, 254 aa, Artificial Sequence
  source: /note=“Description of Artificial Sequence Synthetic polypeptide”
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 3 - DNA, 483 bp, Artificial Sequence
  source: /note=“Description of Artificial Sequence Synthetic polynucleotide”
atgtaccggatgcagctgctgagctgtatcgccctgtctctggccctcgtgaccaacagcgcccctaccagcagcagcaccaagaaaacccagctgcagctggaacatctgctgctggacctgcagatgatcctgaacggcatcaacaactacaagaaccccaagctgacccggatgctgaccttcaagttctacatgcccaagaaggccaccgaactgaaacatctgcagtgcctggaagaggaactgaagcccctggaagaagtgctgaacctggcccagagcaagaacttccacctgaggcccagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgaccggctccgagaaggacgagctgtga

SEQ ID NO: 4 - protein, 160 aa, Artificial Sequence
  source: /note=“Description of Artificial Sequence Synthetic polypeptide”
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL